(19) United States
(12) Patent Application Publication
(10) Pub. No.: US 2015/0246176 A1
(43) Pub. Date: Sep. 3, 2015

(54) FLUID DELIVERY SYSTEM AND METHODS

(71) Applicant: SWISSINNOV PRODUCT SÀRL, Gland (CH)

(72) Inventors: Thierry Navarro, Gland (CH); Florent Junod, Veigy Foncenex (FR)

(21) Appl. No.: 14/436,411

(22) PCT Filed: Oct. 16, 2013
(86) PCT No.: PCT/IB2013/059393

§ 371 Date: Apr. 16, 2015

(30) Foreign Application Priority Data
Oct. 16, 2012 (IB) PCT/IB2012/055626
Mar. 6, 2013 (IB) PCT/IB2013/000302

(51) Int. Cl. A61M 5/142; A61M 5/172; A61M 5/14

(52) U.S. Cl. CPC A61M 5/14248; A61M 5/1413; A61M 5/172; A61M 2005/14252

(57) ABSTRACT

A fluid delivery device for delivering small quantities of fluid such as insulin to a patient, including a disposable unit having a disposable housing that has one lower part and one upper part, which together form a shell that defines an internal partial toroidal arcuate cavity. The housing fits together with a drive unit. The housing contains an arcuate cylinder for containing fluid to be delivered, a piston movably mounted in the cylinder for driving out fluid to be delivered, an adhesive support for attaching the housing and a cannula that when the housing is attached to a patient is insertable in the patient's skin for delivering fluid. The drive unit is preferably removably mounted on a front of the housing opposite the adhesive support, the removable drive unit having a shape that when fitted complements the shape of the front face of the housing to form with the housing.

### TECHNICAL FIELD

The invention described herein is directed to an ultra small fluid delivery system comprising a fluid pumping device and an associated multi-functional remote control including automatic filling and cannula insertion features. The invention is further directed to a method for administering the fluid to a patient. The fluid delivery system according to the invention is intended to be used in any medical application.

This system is particularly adapted to be used as a subcutaneous or transdermal drug delivery patch adhesively attached to the patient's skin. It is preferably used as an insulin patch pump given that its structure makes it ultra small and very light while being capable to deliver a very small amount of insulin or other drug.

### BACKGROUND OF THE INVENTION

Insulin pumps are widely known in the prior art as an alternative to multiple daily injections of insulin by an insulin syringe or an insulin pen.

A wearable patch pump as described in U.S. Pat. No. 4,525,164 uses an arcuate syringe to provide a small and compact drug delivery device. This patch pump has a wearable frame receiving a removable arcuate syringe with a stem that is actuated by a motor placed on the frame when assembled. The fluid is expelled from a syringe barrel through a needle attached to the syringe extremity by a flexible tube. The syringe is affixed to the internal wall of the frame and maintained with clips.

The first drawback of this patch pump is the coupling between the syringe and the frame that will not allow for precise control of the piston movement within the barrel. The presented clips do not insure proper fixation of the syringe on the frame and the syringe can possibly move during a shock or vibration which are common situations of wearable devices. As a result, the amount of drug is not fully controlled and may lead to serious injury for the patient.

The second drawback is the filling and setup of the system. There is no teaching how to setup the system and manipulate the syringe during assembly with the frame. When the stem is fully outside the barrel, there is no element maintaining it in position and it will most probably fall out of the barrel. This leads to an almost impossibility to fill the syringe properly without the help of specific equipment. In addition, during the manual insertion of the filled syringe on the frame, the operator or patient has to be very careful to not push the stem, otherwise the drug will be expulsed inadvertently.

This known pump has several other drawbacks; the plunger is in two parts making it complex to manufacture. The sealing between the piston and the barrel is made without rubber resulting in high friction for avoiding leakage. The insulin is delivered through a needle that is sharp and stiff reducing the number of possible subcutaneous injection sites.

Another wearable patch assembly is described U.S. Pat. No. 8,137,314. The known delivery device is made of two parts, a durable part and a disposable part, connectively removable. This system has no indication as to how to provide the required accuracy of the drug delivery with an efficient assembly composed of a reduced number of parts especially with an arcuate reservoir.

In EP 2438938A1, an injection device is disclosed. This system has a one-part casing with a curved reservoir. This configuration makes its manufacturability complex and not appropriate for a having a low cost system made of injected plastics especially for mass production. There is also no indication as to how provide a reliable and efficient assembly of the system.

Other wearable patch pumps are described in: U.S. Pat. No. 4,601,707, U.S. Pat. No. 4,886,499, U.S. Pat. No. 5,800,420, US 2008/0215006 and WO2011007356.

Other remotely controlled infusion pumps are described in U.S. Pat. No. 5,376,070, U.S. Pat. No. 5,630,710, U.S. Pat. No. 5,634,778 and U.S. Pat. No. 5,582,593.

A wearable patch with needle inserter is described in U.S. Pat. No. 6,960,192.

Other fluid delivery systems including assembly for filling the fluid reservoir are described in WO 97/23252, US2010/024099 and U.S. Pat. No. 8,025,658.

Those systems address only part of the needs of the patients as they all require the operator or patient to fill the reservoir manually. Insulin is a very sensitive drug such as many others that can be only stored for a long period of time in a recipient made of glass such as a vial or prefilled pen cartridge. The reservoirs of the pumps and patch pumps are disposable elements made of plastic. During setup of the systems, the insulin must be transferred from the glass recipient to the plastic reservoir. This operation is performed manually by using an intermediate component such as a syringe or reservoir connector. Then the operator or patient empties the desired amount of drug from the glass recipient and transfers it into the plastic reservoir. The major drawbacks of this process are that the operator or patient has to manipulate the drug which may result in air trapping, emulsion, incorrect filling amount and possible injury with the needle of the syringe.

The drug transfer is not only a source of possible error and injury but is a cumbersome process requiring training and a high degree of confidence by the operator or patient. This then poses a problem to children, elderly, blind or impaired patients that have difficulties or even impossibility to setup the system.

Another drawback of these known patch pumps is the subcutaneous cannula insertion into the body that is either made manually with an external cannula inserter or automatically with an internal cannula inserter. In the first case, the operator or patient needs to indicate to the remote control when the patch pump is ready with the cannula inserted under the skin. In the second case, the cannula insertion is remotely controlled but the inserter remains in the patch pump which occupies place and makes the downscale of the patch pump difficult.

### SUMMARY OF THE INVENTION

An aim of the present invention is to reduce the size and the production cost of a wearable fluid pumping device in order to make it more convenient to wear 24/7 and more economical. The invention provides an efficient assembly for manufacturing an accurate, reliable and low cost wearable fluid pumping device for mass production.

This aim is achieved by a fluid delivery device for delivering small quantities of a fluid to a patient, comprising a disposable housing that comprises one lower part and one upper part, the lower and upper parts together forming a shell that defines an internal partial toroidal arcuate cavity.

The arcuate cavity receives an arcuate cylinder for containing the fluid or itself forms an arcuate cylinder for containing the fluid, and an arcuate piston is preferably movable inside the arcuate cylinder. The piston can have a support at its bottom which cooperates with at least one support on the upper and/or lower part of the housing.

The piston can also have a reinforced stem at its bottom. At least one of said lower or upper parts forming the shell of the disposable housing preferably has an arcuate wall on one half of its circumference opposite to the arcuate cavity.

The fluid delivery device can also have a removable drive unit comprising means for actuating the piston is attachable to the disposable housing. Said arcuate wall can constitute a support for receiving, fixing and sealing the drive unit to the disposable housing. The disposable housing can have a recess with outer borders defined by the inner side of the piston stem and by a diametral line of the disposable housing for receiving therein the drive unit.

The disposable housing of the fluid delivery device is preferably an overall enveloping housing in the form of a generally flat cylindrical disc with rounded/inclined upper edges and a flat bottom, the drive unit occupying about one half of the top surface of the flat cylindrical disc, and wherein an adhesive support is applied against the flat bottom and projects from the flat bottom as a peripheral rim.

The fluid delivery device can have a straight cannula generally perpendicular to an adhesive support placed under one part/shell of the disposable housing and located towards the downstream end on the said toroidal cavity.

A cannula can be movably mounted in the disposable housing between a first position for delivering fluid to a patient and a second position communicating the cavity with the outside for filling the cavity or a cylinder therein with fluid from an external recipient, the cannula passing through the two parts/shells of the disposable housing. Such cannula cooperates with a septum having therein an aperture.

The drive unit is preferably actuable by remote control.

The lower and upper parts/shells of the disposable housing are fixed by ultrasonic welding, glue or by clipping/snap fit.

The arcuate cavity or a cylinder located in the arcuate cavity for example contains insulin for delivery to a patient.

The above aim is thus achieved by a fluid pumping device comprising a disposable housing containing preferably partial toroidal arcuate cylinder, a preferably part-circular arcuate piston with preferably elliptical section, a cannula, at least one septum, an adhesive support and a preferably removable drive unit comprising an adapted case to be fixed to the disposable housing, including a piston actuator, an electronic control unit, sensors and preferably a rechargeable battery.

In one main aspect, the invention therefore comprises a fluid delivery device for delivering small quantities of a fluid to a patient, comprising a disposable unit comprising a disposable housing and a preferably removable drive unit.

The disposable housing contains a cylinder for containing fluid to be delivered, a piston movably mounted in the cylinder for driving out fluid to be delivered, an adhesive support for attaching the disposable housing to a patient, and a cannula that when the disposable housing is attached to a patient is insertable in the patient's skin for delivering fluid to the patient.

The overall enveloping housing is usually a generally flat cylindrical disc with rounded/inclined upper edges and a flat bottom, the drive unit occupying about one half of the top surface of the flat cylindrical disc, and wherein the adhesive support is applied against the flat bottom and projects from the flat bottom as a peripheral rim.

The piston usually comprises a piston head engaging in the arcuate cylinder and a generally arcuate stem extending from the cylinder, the piston having an elongate stem having thereon s serrated rack engageable with a toothed wheel forming part of the means for actuating the piston.

The disposable housing's cylinder advantageously has an elliptical cross-section with its large section generally parallel to the adhesive support, and the piston has a piston head of corresponding elliptical shape engaged in the cylinder.

Another aspect of the invention is a disposable unit of the fluid delivery device, the disposable unit comprising a disposable housing containing a cylinder for containing fluid to be delivered, a piston movably mounted in the cylinder for driving out fluid to be delivered, and adhesive support for attaching the disposable housing to a patient, and a cannula that when the disposable housing is attached to a patient is insertable in the patient's skin for delivering fluid to the patient, the top face of the disposable housing opposite to the adhesive support having a recess for receiving therein a drive unit to form a fluid delivery device.

A further aspect is the removable drive unit of fluid delivery device, the drive unit being removably mountable on a front face of the fluid delivery device's disposable housing opposite the adhesive support, the removable drive unit having a shape that when fitted complements the shape of the front face of the disposable housing to form with the disposable housing an overall enveloping housing for the fluid pumping device, the drive unit comprising means for actuating the piston and a control unit for the device.

The drive unit is mounted, preferably removably, on a front face of the disposable housing opposite the adhesive support, the mounted drive unit having a shape that when fitted complements the shape of the front face of the disposable housing to form with the disposable housing an overall enveloping housing for the fluid pumping device, the drive unit comprising means for actuating the piston and a control unit for the device.

Further aspects consist on the one hand of the disposable unit as defined and, on the other hand, the removable drive unit as defined.

Yet another aspect of the invention is the combined automatisation of the filling of the reservoir in order to simplify the system setup, reduce the risk of errors, making it more convenient for children, elderly, blind or impaired persons and the automatisation of the cannula insertion into the body.

This is achieved by a removable bi-functional connector attached to the disposable housing, comprising a drug recipient support, a movable needle, a needle grip, an optional needle actuator.

The bi-functional connector is removably fittable on the fluid delivery device. In one embodiment, it comprises a support for a fluid recipient, a movable needle held in a needle grip, the needle being movable when the bi-functional connector is fitted on the fluid delivery device between a position for delivering fluid from the fluid recipient to the cylinder, a position for causing the needle to pierce the patient's skin for insertion of the cannula and a position allowing the cylinder to deliver fluid via the cannula.

Another aspect of the present invention is to provide an associated multi-function remote control that is adapted to communicate wirelessly with the fluid delivery device, the remote control comprising a plurality of controls for different functions of the fluid delivery device. The remote control is for example adapted to automatically fill the fluid recipient, automatically insert the cannula into the body, integrate a glucose sensor and a lancing device, making the remote control an “all-in-one” component in order to reduce the number of separate devices to manage diabetes.

This is achieved by a portable device communicating wirelessly with the fluid delivery system comprising an electronic control unit, a display, an optional keypad, sensors, an optional glucose sensor, an optional test strips compartment, an optional needle release mechanism, an optional lancing device, an optional lancets compartment.

Advantageously, the wireless-operated remote control comprises a plurality of controls for different functions of the fluid delivery device including controls for: automatically filling the cylinder with fluid and automatically inserting the cannula into a patient's body, the remote control comprising an electronic control unit, a display, an optional keypad, and at least one sensor-actuated control.

An even further aspect of the present invention is to provide a method for setting up the fluid device and insert the cannula into the body. This method comprises: adhering the disposable housing to a patient's skin by means of the adhesive support; fitting a bi-functional support on the fluid delivery device, the bi-functional support carrying a recipient of fluid to be delivered and being adapted to deliver fluid to the fluid delivery device; actuating the bi-functional support to deliver fluid to the disposable housing's cylinder; and actuating the needle to pierce the patient's skin for inserting the cannula and bring the cannula into communication with fluid in the cylinder for delivering fluid to the patient's body.

Further aspects of the invention are set out in the detailed description and in the claims.

### BRIEF DESCRIPTION OF THE DRAWINGS

The invention will be better understood thanks to the following detailed description of several embodiments with reference to the attached drawings, in which:

FIG. 1 is a perspective view of the fluid pumping device.

FIG. 2 is a perspective view of the fluid pumping device's disposable housing optionally with a breakable element used for indicating that the disposable housing has already been used.

FIG. 3a is an outside side view of a drive unit.

FIG. 3b is a bottom view of the drive unit with electrical connector plus a motor gear wheel.

FIG. 4a is a top plan view of the fluid pumping device's disposable housing.

FIG. 4b is a section view along line A-A of FIG. 4a.

FIG. 5a is a schematic side elevation of the fluid pumping device's disposable housing.

FIG. 5b is a section view along line B-B of FIG. 5a,

FIG. 6 is an open view of the upper and lower parts/shell of the disposable housing case forming a cavity for receiving the cylinder.

FIG. 7 shows a preferred arcuate cylinder with septum support at a lower end part of the cylinder.

FIG. 8 shows a preferred arcuate piston with piston head receiving at least one O-ring or at least one dedicated gasket as sealing element with the cylinder.

FIG. 9 shows a cannula with lateral hole and stopper head including a septum to be fixed in the cylinder support at bottom position.

FIG. 10 shows a septum with hole for connecting the cannula with the cylinder inlet/outlet port.

FIG. 11 is a perspective view of the disposable housing partly without its upper case part/shell.

FIG. 12a is a schematic diagram of the disposable housing showing section lines C-C, D-D and E-E.

FIG. 12b is a section view along line C-C of FIG. 12a.

FIG. 12c is a section view along line D-D of FIG. 12a.

FIG. 12d is a section view along line E-E of FIG. 12a.

FIG. 13a is a schematic side elevation of the fluid pumping device with the drive unit fitted on the disposable housing.

FIG. 13b is a section along line F-F of FIG. 13a.

FIG. 14 is a perspective view of the disposable unit with the bi-functional connector attached.

FIG. 15 is a rear perspective view of the disposable unit with the bi-functional connector attached and a drug recipient fitted.

FIG. 16a is schematic plan view of the disposable unit with the bi-functional connector attached and drug recipient fitted.

FIGS. 16b, 16c and 16d are section views along line G-G of FIG. 16a with the bi-functional connector attached to the disposable housing and drug recipient fitted, showing operative parts in three different positions.

FIG. 17a is a schematic side elevational view of the disposable unit with the bi-functional connector attached and drug recipient fitted.

FIG. 17b is section view along line H-H of FIG. 17a.

FIG. 17c is a section view along line I-I of FIG. 17a, wherein a bi-functional connector has optionally a second needle fixed on it.

FIG. 18a is a front view of a first embodiment of an “all-in-one” multi-function remote control.

FIG. 18b is a back view of the first embodiment of “all-in-one” multi-function remote control.

FIG. 19 is a perspective view of the lancing device that is fitted on the back of the multi-function remote control, having preferably at least one compartment for disposable lancets and preferably one compartment for disposable glucose strips.

FIG. 20 is a perspective view of a second embodiment of multi-function remote control having a preferred removable control unit with a preferred touch screen.

FIGS. 21 and 22 show another embodiment of a multi-function remote control showing a control unit fitted and removed.

FIG. 23 is a perspective view of another remote control with an optional lancet compartment and an optional test strip compartment.

FIG. 24 shows another removable lancing device with at least one optional compartment.

FIGS. 25a, 25b, 25c and 25d are perspective views showing a second embodiment of the bi-functional connector.

FIG. 26 is a diagram of the electronic circuitry of the remote control and/or the drive unit.

FIG. 27 is a flow diagram of successive steps in a method of implementation.

FIG. 28 is a view of a disposable patch with a centered cannula.

FIG. 29 shows the patch of FIG. 29 in side elevation.

FIG. 30 is a plan view of the patch of FIGS. 28 and 29, drive unit removed.

FIGS. 31 and 32 show the two parts/shells of a patch making up the disposable housing and defining the inner arcuate cavity that is assembled from the two parts/shells.

FIG. 33 is a bottom view of the piston and drive unit with the motor gear at 90°.

FIG. 34 is a perspective view of another embodiment of the disposable housing with a central axis for guiding the piston.

FIG. 35 is perspective exploded view of another embodiment of the disposable housing.

FIG. 36 is a perspective view of another embodiment of the piston with a support and dedicated gasket.

FIG. 37 is a side view of another embodiment of the movable needle.

### DETAILED DESCRIPTION

### The Fluid Pumping Device

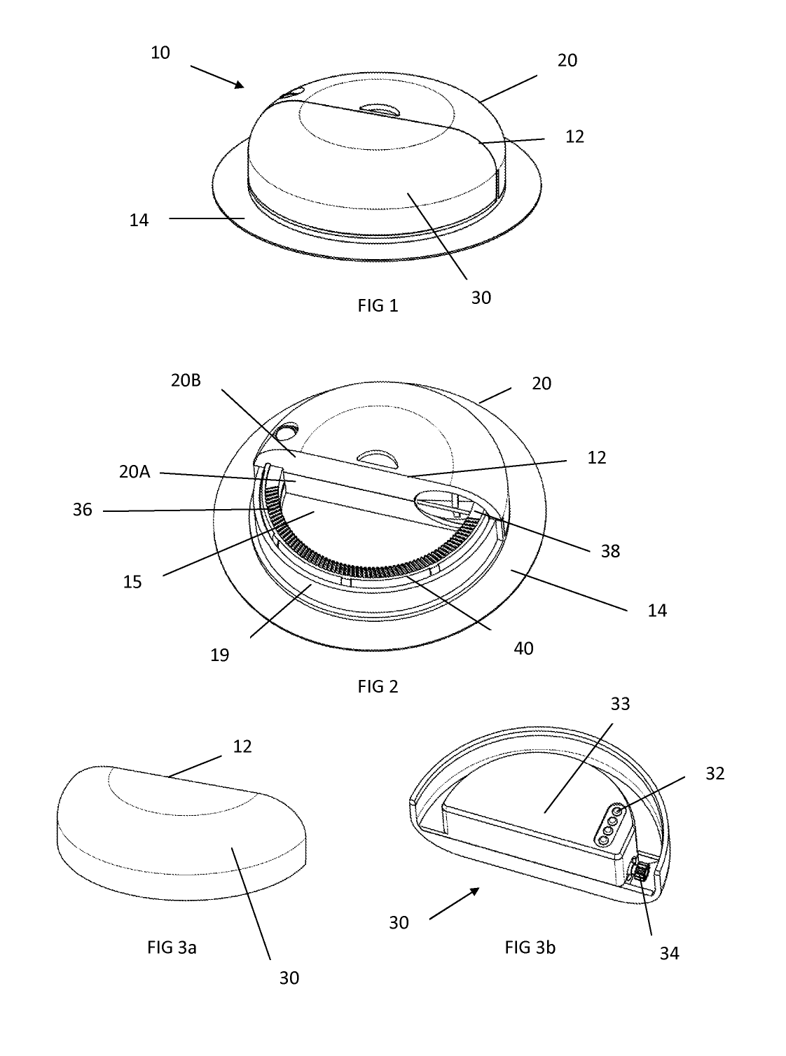

The fluid pumping device 10 shown in FIG. 1 comprises a disposable housing 20 shown in FIG. 2 and a drive unit 30 shown in FIGS. 3a,3b. As shown, the overall enveloping housing is a generally flat cylindrical disc with rounded/inclined upper edges and a flat bottom, the drive unit 30 occupying about one half of the top surface of the flat cylindrical disc, divided by a diametral dividing line 12. A sheet-like adhesive support 14 is applied against the flat bottom and projects from the flat bottom as a peripheral rim.

The device optionally has a breakable element for indicating that the disposable housing has already been used.

The bottom part/shell of the disposable housing 20 has adhesive means for the disposable housing to be fixed on the patient's skin, namely the flat support 14 with adhesive on its lower surface protected by a removable peel-off layer.

The drive unit 30 shown in FIGS. 3a and 3b has preferably an electrical connector 32 and a motor gearwheel 34 that engages with serrations 36 on the piston, see FIG. 2, when the drive unit 30 is fitted on the disposable housing 20. The gear wheel 34 is preferably located outside the drive unit case 33 to engage externally with serrations 36.

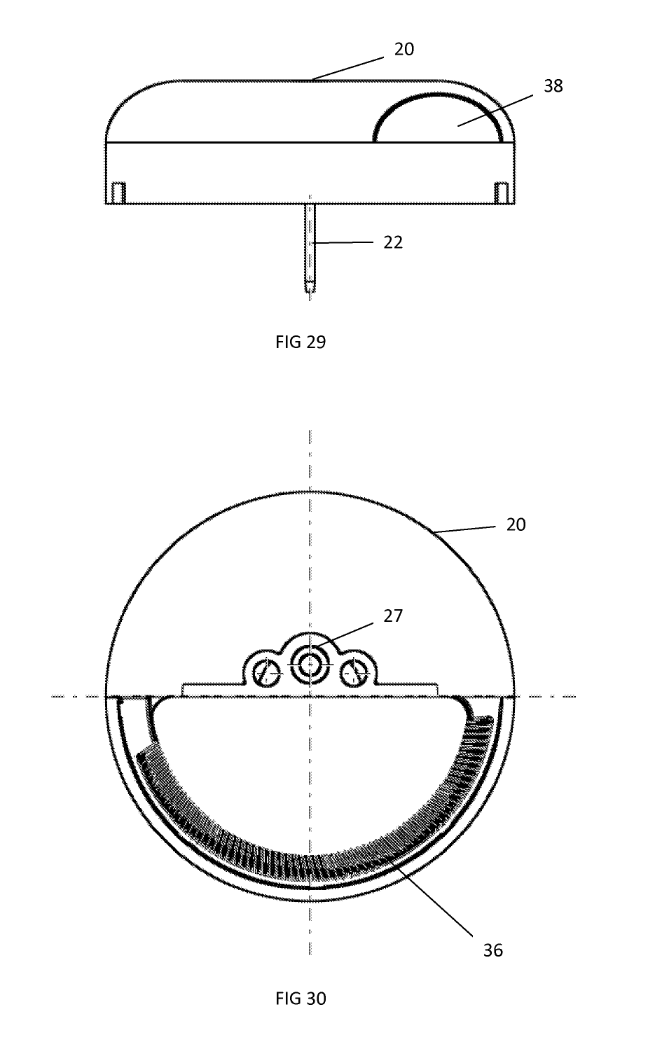

The disposable housing 20 comprises towards its periphery a cannula 22. The cannula 22 is straight and is preferably perpendicular to the adhesive support and as shown is located preferably towards the periphery of the disposable housing 20 at the downstream end of the cylinder 28. Alternatively, the cannula 22 can be located centrally (FIG. 29).

FIG. 2 shows the assembled disposable housing 20 formed by an upper part/shell 20B and a lower part/shell 20A forming a preferably toroidal arcuate cavity wherein the piston 38 can move inside. The lower part/shell 20A has preferably an arcuate wall 19A (FIG. 34) on one half of its circumference opposite to the arcuate cavity 13. The wall 19A is preferably used to support and guide the piston 38, 138. The disposable housing 20 has a recess 15 with preferable outer borders defined by the inner side of the piston stem 40 and the diametral line 12 for receiving therein the drive unit 30. The wall 19A is also preferably used as support for receiving, fixing and sealing the drive unit 30 to the disposable housing 20.

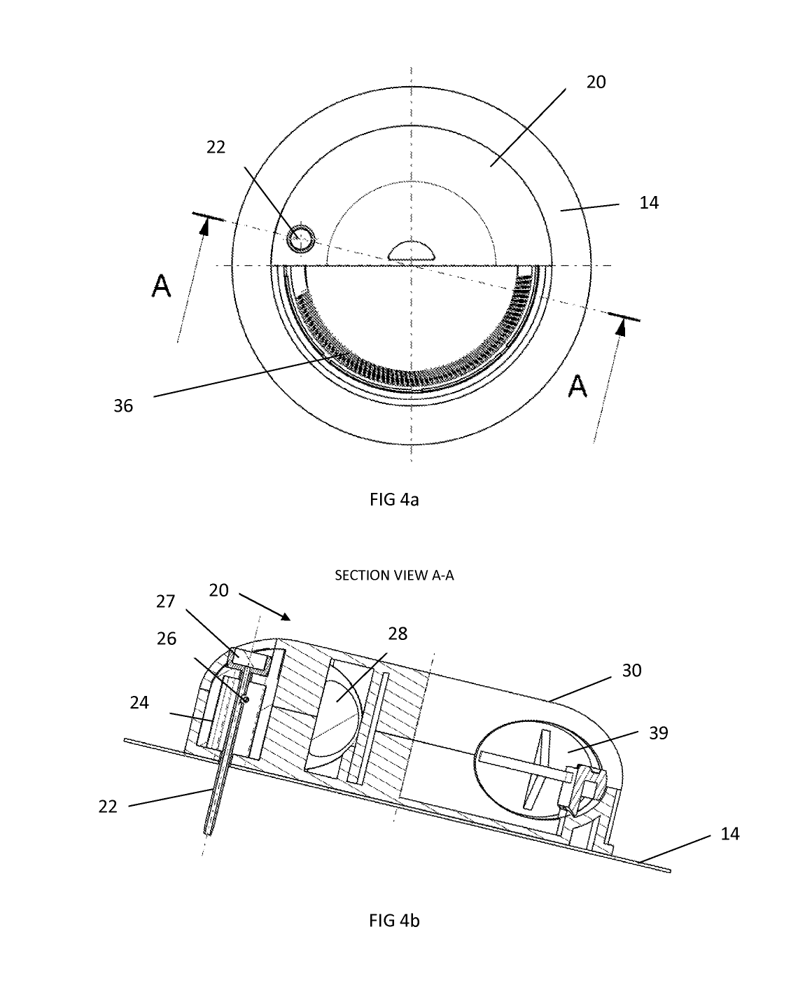

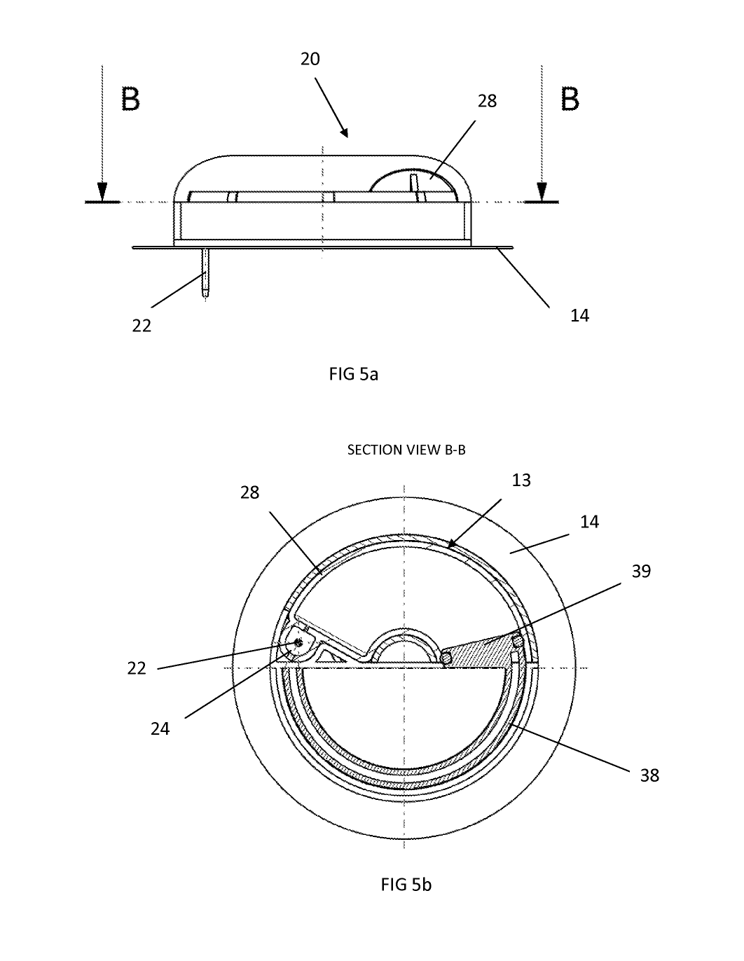

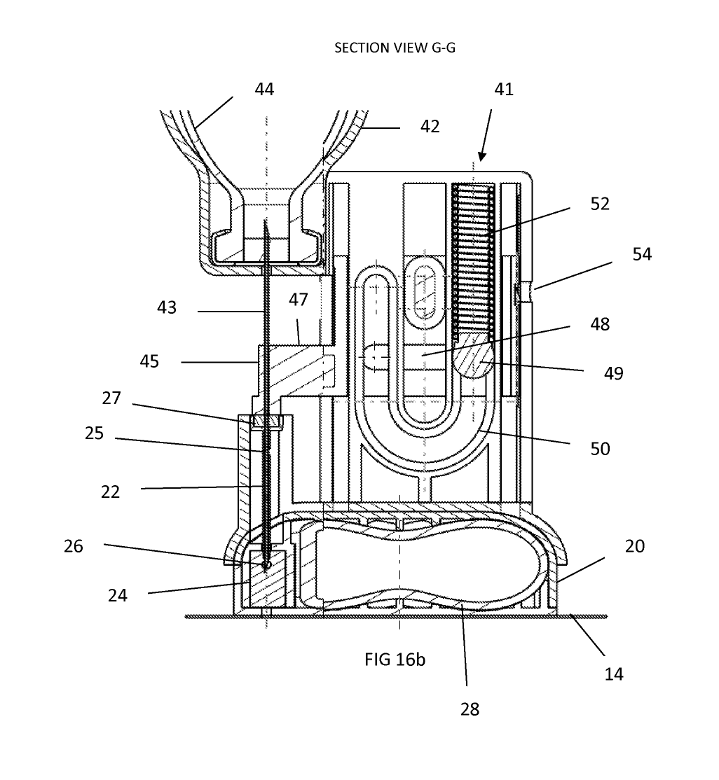

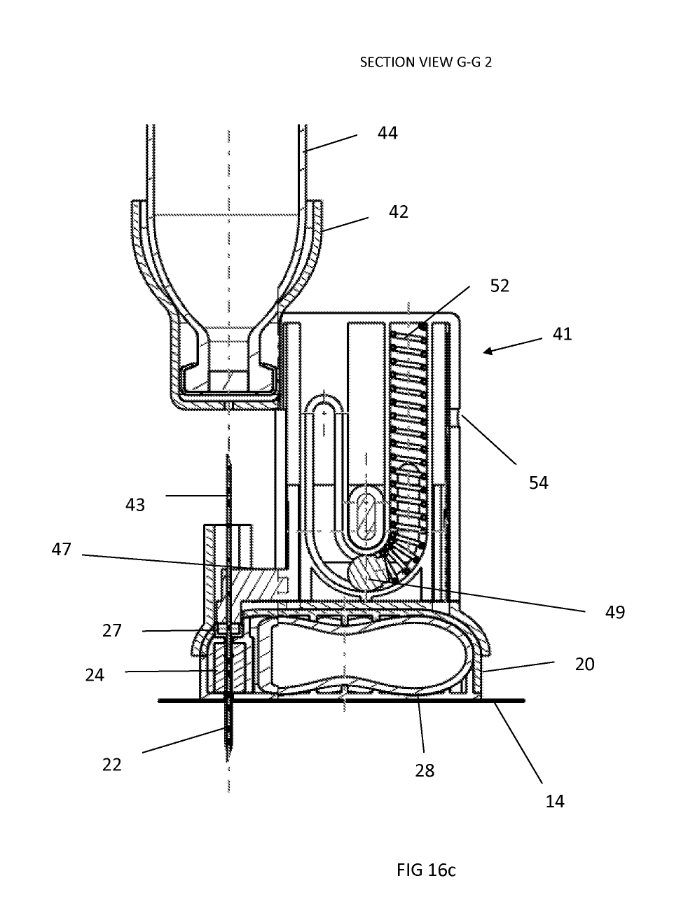

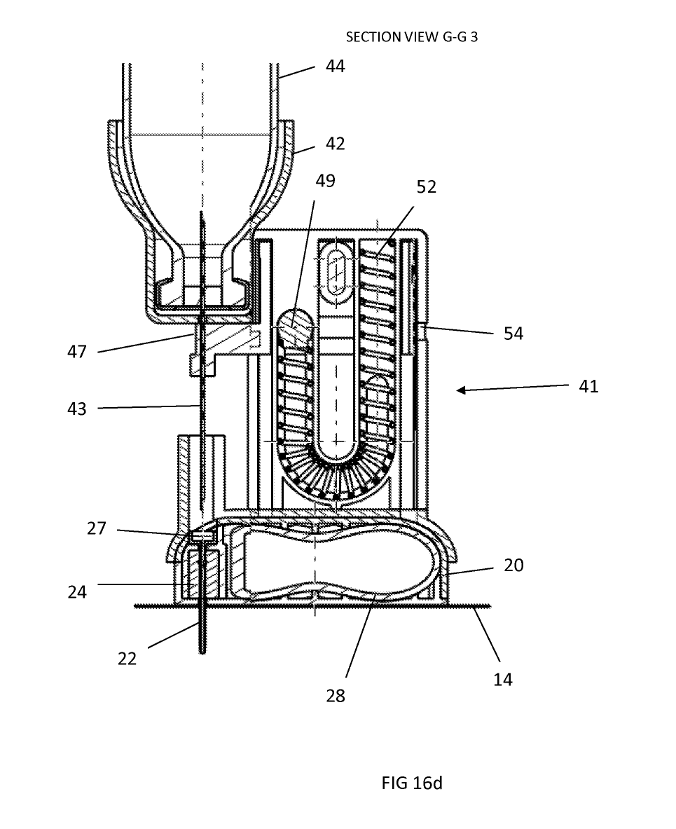

FIG. 4b shows the cannula 22 at its bottom position inside a septum 24 and in communication, via a through hole 26 in the septum 24, with an arcuate cylinder 28. The cannula 22 is movably mounted between a first position (FIG. 4b) for delivering fluid to a patient and a second position (FIG. 16b) communicating the cylinder 28 with the outside for filling the cylinder with fluid from an external recipient 44.

FIG. 5b shows a preferably arcuate cylinder 28 positioned inside the disposable housing 20, with the piston 38 positioned in its end position inside its cylinder, and the septum 24 positioned at an end part of the cylinder 28.

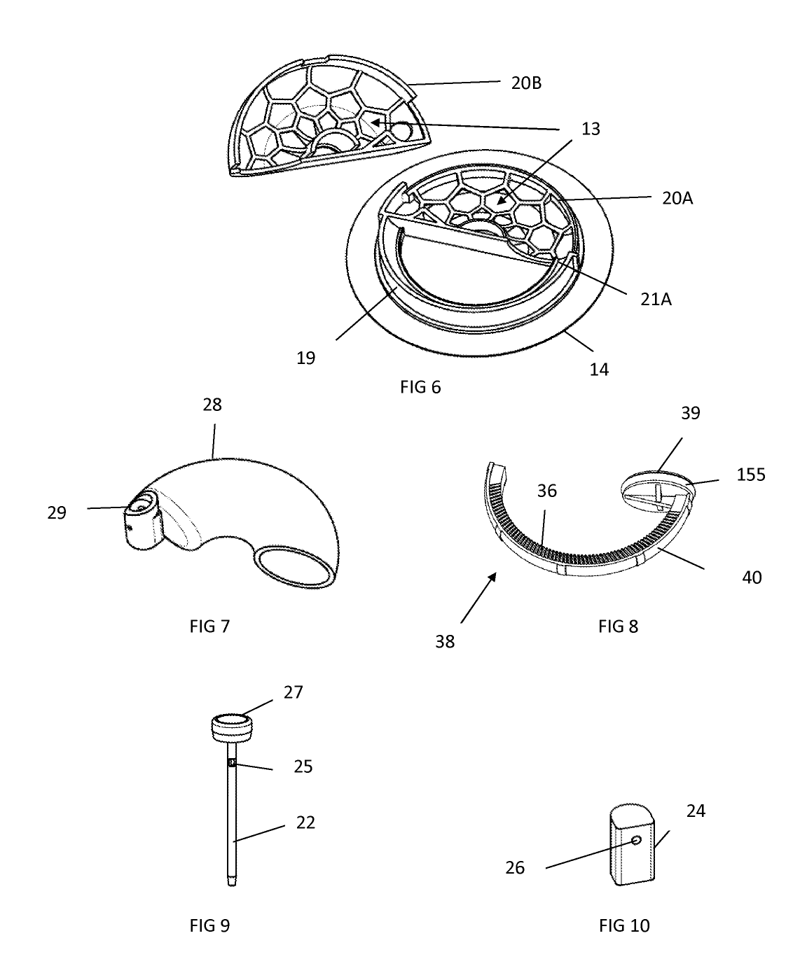

FIG. 6 is an open view of the upper and lower parts/shell 20A and 20B of the disposable housing 20's case forming preferably a partial toroidal arcuate cavity 13 for receiving the cylinder 28. The inner side of the wall 19A has preferably a curved profile 21A adapted to the cross sections of the arcuate cavity 13 and cylinder 28 allowing the piston stem 40 to move properly along the cylinder while being in contact with wall 19A.

FIG. 7 shows the disposable housing's cylinder 28 which as shown is preferably an arcuate cylinder with a septum support 29 at the downstream end part of the cylinder 28, i.e. at its left hand end. See also FIG. 5b where the arcuate cylinder 28 is fitted in the disposable housing 20.

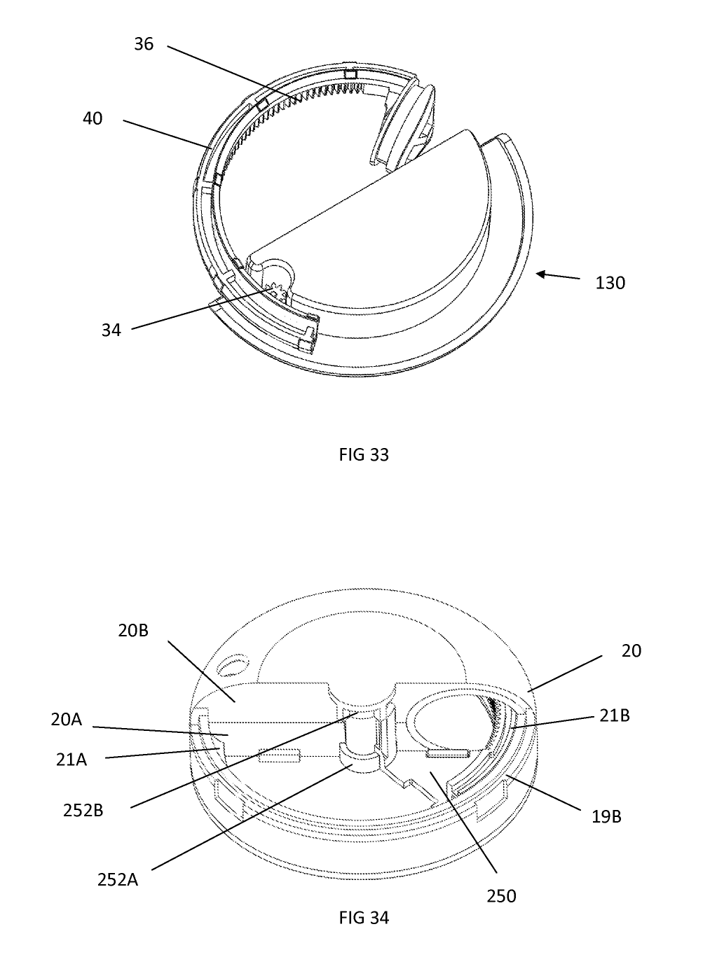

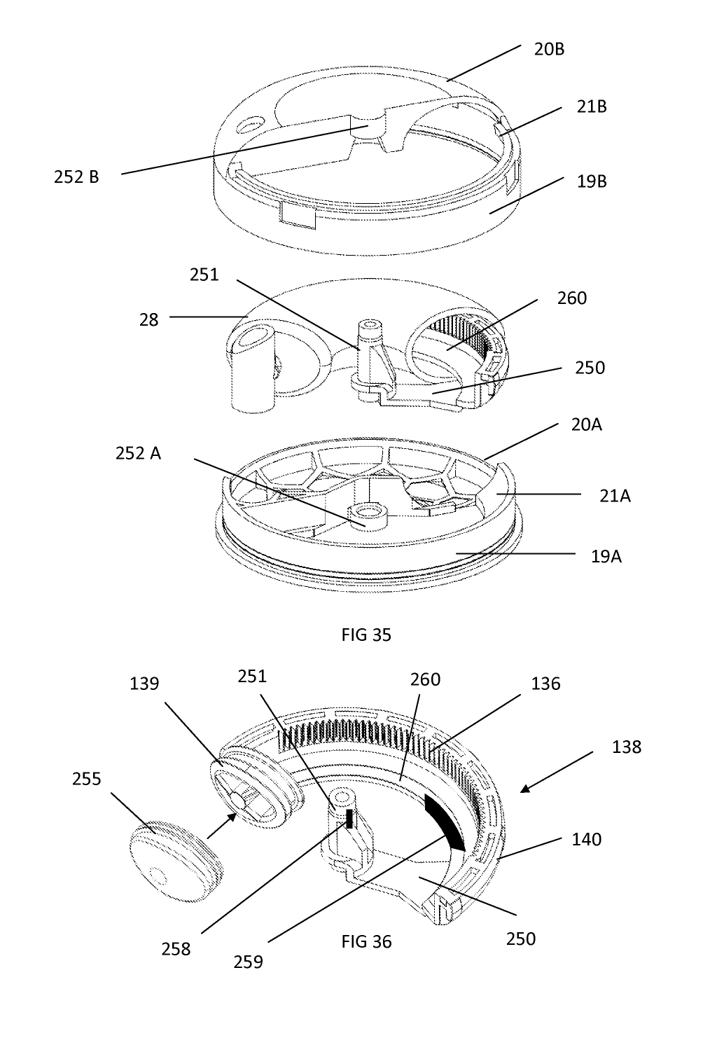

FIGS. 8, 35 and 36 show the piston 38, 138 which as shown is preferably an arcuate piston corresponding to the arcuate cylinder 28. As shown, the arcuate piston 38 has a piston head 39, 139 receiving at least one O-ring 155 or at least one dedicated gasket 255 as sealing element with the cylinder 28. This piston head 39 has an arcuate stem 40, 140 with a corresponding elliptical shape to the inner part of the arcuate cylinder 28. The piston stem 40, 140 has a rack 36, 136 with a series of serrations preferably on the upper side, as shown. The rack 36, 136 has a special shape to be engaged with the gearwheel 34 of the drive unit, i.e. motor 31. The piston shape has a special design as the axis of motor 31 is eccentric from the center of the disposable housing 20. The bottom part of the piston head 39, 139 is reinforced with the stem to avoid bending. In another embodiment shown in FIGS. 33, 36 the rack 36, 136 is located on the inner side of the stem 40, 140 and the motor gearwheel 34 is positioned at 90° to be engaged with the rack 36, 136. Supports are placed along the stem to provide a better guiding while reducing the friction with the disposable housing and cylinder.

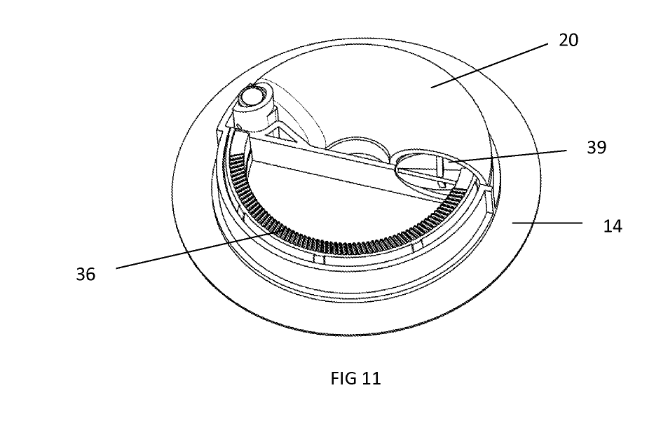

The cannula 22 cooperates with a septum 24 having therein an aperture 26. FIG. 9 shows the cannula 22 with a lateral hole 25 and stopper head 27 including a septum to be fixed in a cylinder support at bottom position. FIG. 10 shows the septum 24 with its hole 26 for connecting the cannula with the cylinder inlet/outlet port.

FIG. 11 shows the disposable housing 20 with its upper case part/shell partly cut away.

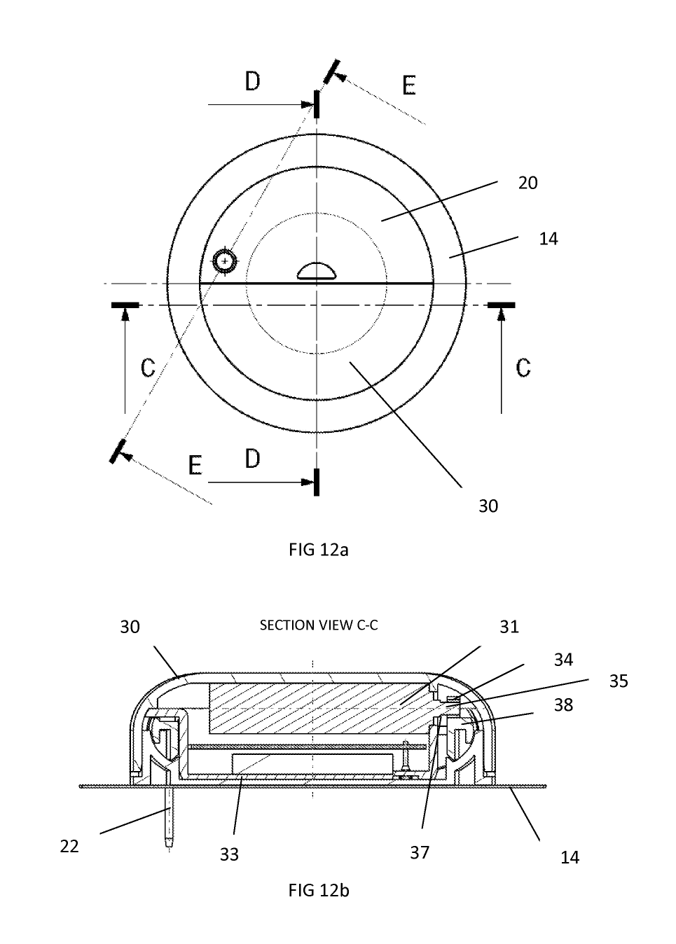

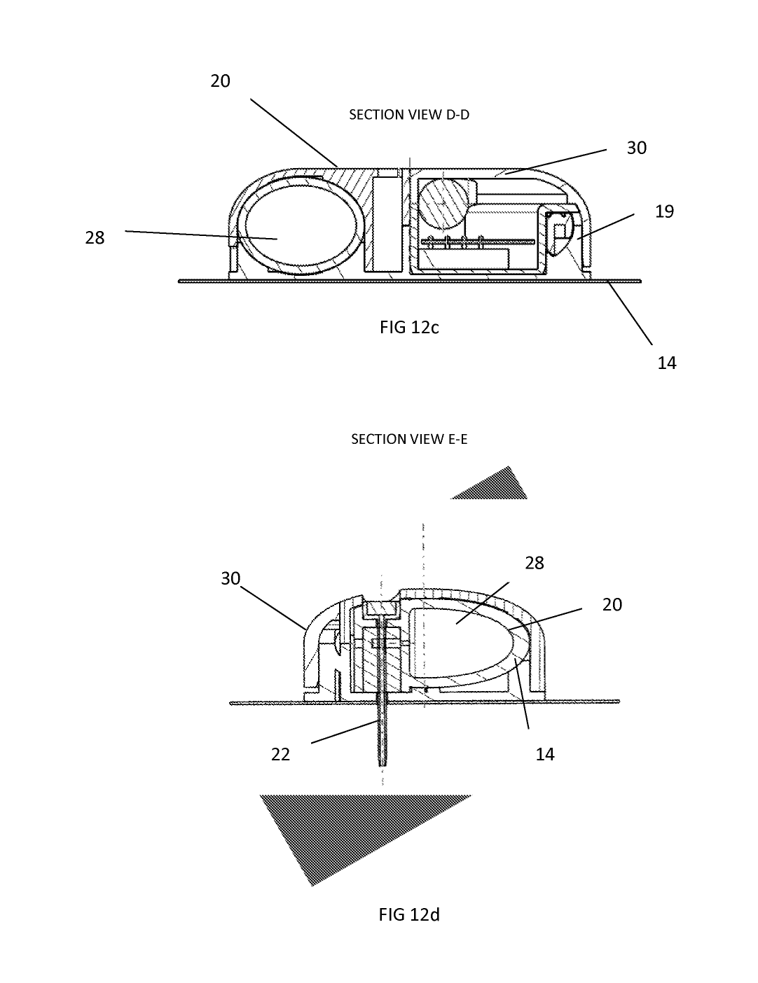

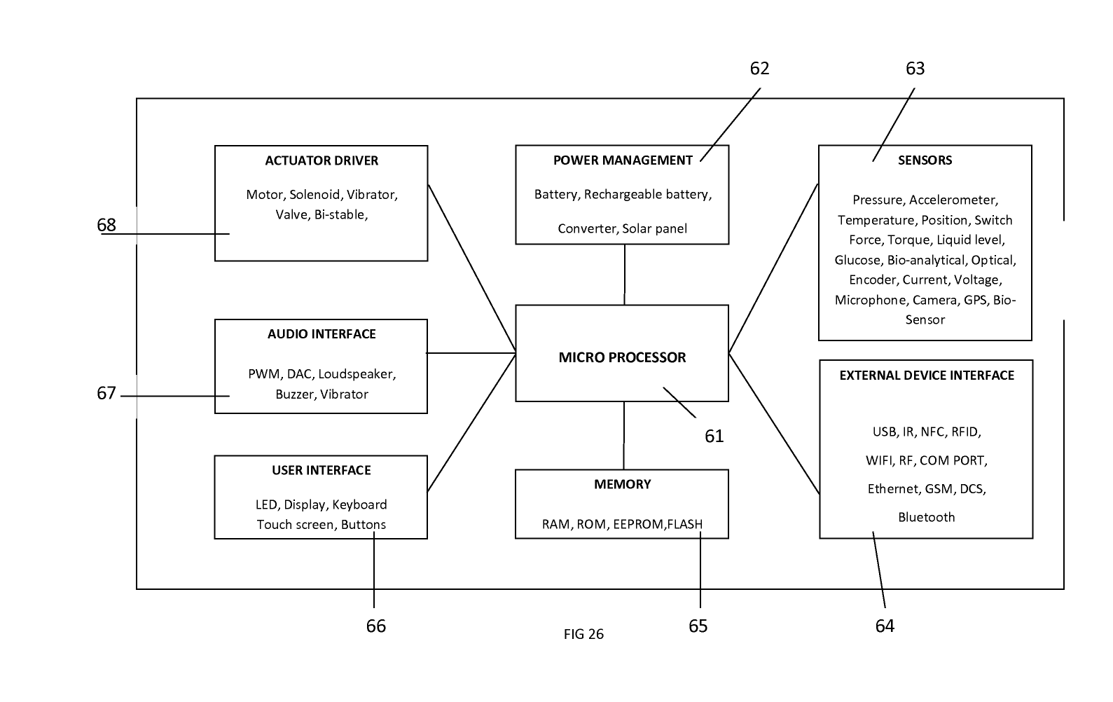

FIG. 12b's section view shows the drive unit 30 coupled with the disposable housing 20, and the motor gearwheel 34 engaged with the piston stem's rack 36. Rotation of the motor gearwheel 34 moves the piston 38 whose piston head 39 is guided along the disposable housing. As a result, liquid is expulsed via the cylinder inlet/outlet port and through the cannula 22 under a patient's skin. The motor 31 is driven by an electronic control (FIG. 26) and powered preferably with at least one rechargeable battery. The motor axis 35 passes through an opening 37 in the drive unit case 33 to receive gearwheel 34 outside of the drive unit case 33. A sealing element not illustrated preferably such as an O-ring is positioned in the opening 37 in the drive unit case 33 to make the drive unit air and water tight.

FIG. 12c is a section view along line D-D of FIG. 12a showing the cylinder 28's section which as shown is preferably elliptical to reduce the height of the reservoir, the long diameter of the ellipse being parallel to the plane of the support 14.

FIG. 12d is a section view along line E-E for FIG. 12a, showing the cannula 22 at its bottom position, wherein the cylinder inlet/outlet port is in communication with the septum hole 26 and with the cannula hole 25. The cannula head 27's septum insures sealing to ambient air on the upper side of the cannula 22.

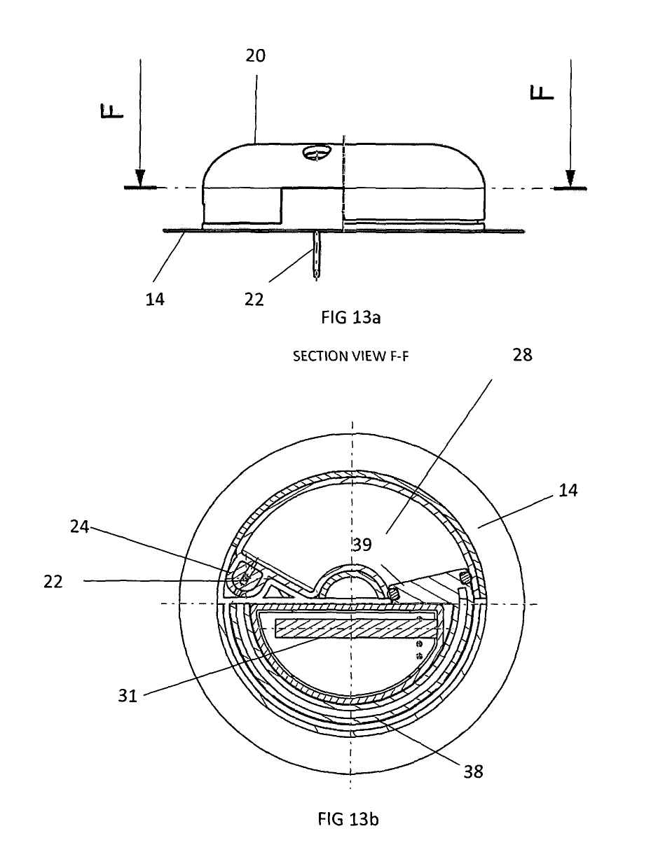

FIG. 13b is section view along line F-F of FIG. 13a showing how the motor 31 is preferably placed eccentrically to the disposable housing to reduce the size of the drive unit 30 allowing for a larger cylinder volume. The motor 31 is also preferably placed closer to the cylinder upper part (aperture) to have the best piston guiding.

FIGS. 34, 35 and 36 are perspective views of a preferred embodiment of the piston 138 having a support 250 preferably engaged in the center of the disposable housing by means of an element such as a joint, hinge, bearing or any rotatable element 251 attached to at least one fixed support 252A and/or 252B respectively part of the lower and upper parts/shells 20A and 20B of the disposable housing 20, allowing the piston to be axially guided with the disposable housing 20. The use of a central guiding assembly improves the piston displacement accuracy during the release of the drug to the patient. It also reduces the friction of the piston along the disposable housing. The piston stem 140 has at its bottom a reinforcement 260 preferably extending along the entire stem. This stem reinforcement 260 serves for rigidifying the piston 138, avoiding the stem 140 to bend and ensuring a good accuracy of the fluid delivery device 10.

The piston support 250 is preferably positioned at the bottom of piston 138 and at the bottom of the rotatable element 251 close to the support 14, opposite to the recess's opening 15 receiving the drive unit 30. The positioning of the piston support 250 and stem reinforcement 260 at the bottom of the piston 138 and rotatable element 251 allows the piston 138 to move freely under the drive unit 30 when the drive unit 30 is placed in the disposable housing 20.

The upper part/shell 20B has preferably an arcuate wall 19B (FIG. 34) on one half of its circumference opposite to the arcuate cavity 13. The inner side of the wall 19B has preferably a curved profile 21B adapted to the cross sections of the arcuate cavity 13 and cylinder 28 allowing the piston stem 140 to move properly along the cylinder while being in contact with wall 19B. The walls 19A and 19B are adapted to form, when in contact, a smooth continuous cross section profile for avoiding vertical movement of the piston stem 140 during the rotational movement of the piston 138 horizontally. The wall 19B preferably overlaps the wall 19A when the upper and lower parts/shells 20B and 20A are attached together. The wall 19B is also preferably used as support for receiving, fixing and sealing the drive unit 30 to the disposable housing 20.

The piston support 250 or any part of the piston can optionally have at least one sensor element 258,259 preferably made of metal, ferromagnetic, or plastic compound having a special shape or profile. This sensor element 258, 259 is preferably placed such as to be close at least to one inductive, capacitive, magnetic, optical or mechanical fixed sensor, located in the drive unit 30, 130. The relative movement of the sensor element 258, 259 with the fixed sensor(s) in the drive unit, allows measuring precisely the piston displacement during operation. The measured displacement is processed by the electronic circuitry of the drive unit in order to detect displacement errors, piston blockage, fluid delivery occlusion, start and stop positions, or any other relevant positioning information. Such information can also be used as positioning feedback to control the piston movement in a closed loop.

The sensor element above described can also be directly any part of the piston. As for example, the piston stem can be made of metal, magnetic material, or a plastic compound charged with metal, magnetic or mineral particles.

### The Electronic Circuitry

As stated above the fluid pumping device's motor 31 is driven by an electronic control and it is powered preferably with at least one rechargeable battery. The electronic circuitry has optionally one electrical, magnetic or optical sensor for controlling the piston position.

FIG. 26 is a diagram of one embodiment of the electronic circuitry of a multi-function remote control 70, the drive unit 30 or control unit 82, 92. As shown, the circuit includes a CPU (micro processor) 61 functionally connected to:
A power management unit 62 for managing electrical supply from a rechargeable or non-rechargeable battery, an ac-dc, dc-ac converter or a solar panel.
A sensor unit 63 grouping preferably together a pressure sensor, accelerometer, etc as shown.
An external device interface 64 using different communication protocols as indicated.
A memory, 65 which can be RAM; ROM, EEPROM or Flash.
A user interface 66 including optionally LED, Display, touch screen and various control buttons or keyboard.
An optional audio interface 67.
An actuator driver 68 notably for the device's electric motor 31, or solenoid, vibrator, valve or bistable.

Each device—the remote control 70, the drive unit 30 or control unit 81, 92—can contain all of the components of the electronic circuit, or only a part. It is also possible for security reasons to provide a second CPU, for example one in the remote control or the control unit and one in the drive unit.

The electronic circuit typically includes a CPU, a memory, a motor driver, power management, optionally a vibrator, optionally a sound speaker, optionally a visual indicator, optionally a temperature indicator, optionally a humidity sensor, a wired or wireless communication interface to transmit and receive data with external devices such as a remote control, smartphone, tablet, PC, glucose sensor, bio-analytical sensor, bio-sensor or any other type of electronic device, and optionally a sensor to detect the status of the breakable element of the disposable housing. The memory can store preset information such as bolus and basal rates for delivering drug at pre-programmed period of time. The electronic circuitry can receive orders from external device to deliver the drug on demand. The electronic circuitry can be programmed remotely with any type of program of drug delivery profile optionally combining user input and sensors data such as for example glucose level. The electronic circuitry can be adapted to work in a closed loop with glucose sensor or any other bio and bio-analytical sensor.

Multi-axis accelerometer sensors are optionally integrated to the electronic circuitry for sensing shocks, driving position, and for measuring patient activity.

The electronic circuitry can be adapted to sense occlusions in drug pathway by measuring motor parameters such as current and voltage in order to calculate torque at the motor gearwheel that is adapted to the force applied to the piston.

A sensor such as a strain gauge or flexion sensor is optionally positioned in contact with the motor to sense the motor displacement resulting from high torque during occlusion.

All data collected by the sensors, motor commands, threshold values and system status can be stored and reprogrammed in the memory unit 65 and transmitted to the external device for storage, processing of data, activation of procedures, closed loop control and system supervision.

Firmware and software of the drive unit 30, remote control 70 or control unit 82, 92 can be updated by means of the external device under certain conditions. Such update is preferably done through a procedure including user input such as an optional secure code.

A temperature sensor, humidity sensor or accelerometers can detect unsecure conditions such as overheating, water infiltration, shocks that could possibly alter the correct working conditions of the drive unit. The electronic circuitry is adapted to manage alerts when normal conditions are not met and stop the system, emit alerts through a vibrator, sound speaker or visual indicator, and transmit alert information to an external device.

A vibrator, sound speaker or visual indicator can also give feedback to the user on system status, failure or order confirmation.

A watch dog can be used to supervise the circuitry activity and detect any abnormal situation such as for example but not limited to CPU errors, sensor faults, PCB problems or battery failure.

The communication between the drive unit 30 and the remote control 70 or the control unit 82 can be done by using a low energy protocol such as for example NFC (Near Field Communication). The remote control 70 or the control unit 82 will preferably provide the energy for reading and transmitting the data in order to avoid using power from the drive unit's battery.

The Disposable Unit with Bi-Functional Connector Attached

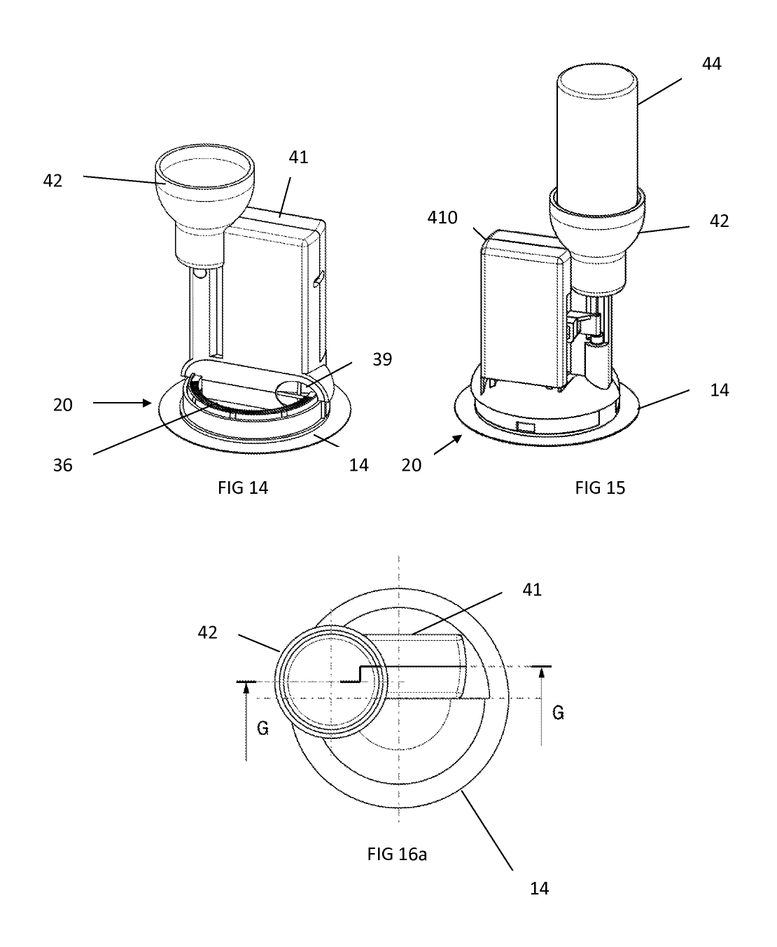

FIG. 14 is a view of the disposable unit 20 with the bi-functional connector 41 attached and FIG. 15 further shows the drug recipient 44 fitted in a support 42.

FIGS. 16b, 16c and 16d are section views along line G-G of FIG. 16a showing the bi-functional connector 41 attached to the disposable housing 20 and with the moving parts in different positions. The bi-functional connector's needle 43 is fixed on a movable needle grab/clamp 45. The needle 43 is preferably open on both extremities, the upper extremity passes through the drug recipient 44's septum and is in contact with the stored drug. The drug recipient 44 can be a prefilled cartridge, vial or any other type of recipient. In the position of FIG. 16b, the second, lower needle extremity faces the disposable septum hole 26. The needle 43 makes a pathway between the drug and the arcuate cylinder's inlet/outlet port through the septum hole 26. The cannula 22 is at an upper position around the needle 43. The cannula hole 25 is closed by the needle body. The needle 43 passes through the cannula head septum and traverses the whole cannula 22.

The needle grab/clamp 45 is connected to a movable support 47 having a horizontal groove 48. A pin 49 is engaged inside the groove 48 and placed in a U-shape groove 50 that is part of the bi-functional connector's case. A compression spring 52 is placed in one arm of the U-shape and is compressed between the pin 49 and one extremity of the U-Shape. The movable support 47 is maintained in position with a holder placed on the inner side of the bi-functional connector's case. An aperture 54 on the side of the bi-functional connector's case allows an external element to push the movable support 47 or the holder to disengage the movable support from the holder. Then the spring 52 pushes the pin inside the U-Shape that activates the movable support 47 to move axially in the direction of the disposable housing 20 during the pin's displacement down in the first half of the U-Shape (FIG. 16c) and in the opposite direction during the pin's displacement up in the second half of the U-Shape (FIG. 16d). The imparted to-and-fro movement of the needle 43 by the movable support pushes the needle 43 in the direction of the patient's skin, piercing it as shown in FIG. 16c and in the same time pushes the cannula 22 with the needle 43. Once arrived at the bottom position, the cannula head 27 is fixed in the cylinder support. When the movable support 47 moves back, the needle 43 goes outside the cannula 22 and is free from the disposable housing 20, as shown in FIG. 16d. The cannula 22 is then placed inside the patient's skin and the cannula hole communicates with the cylinder inlet/outlet port through the septum hole 26.

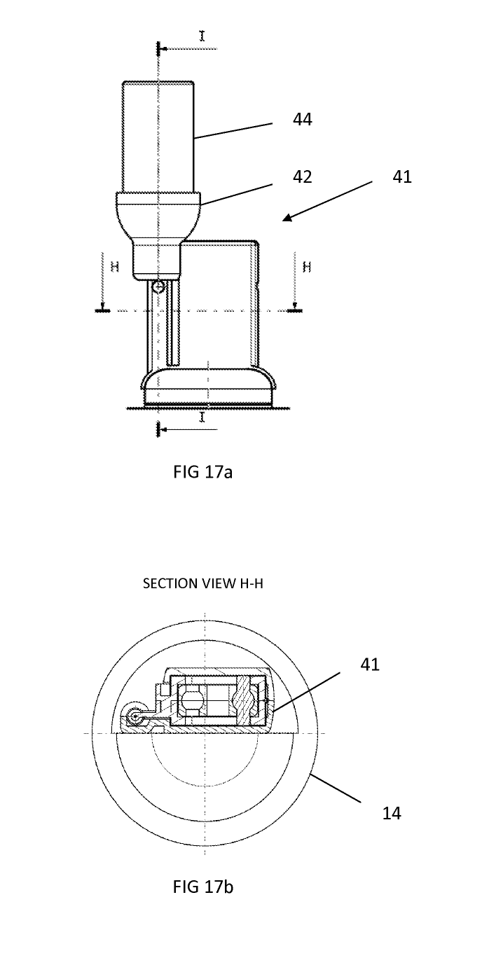

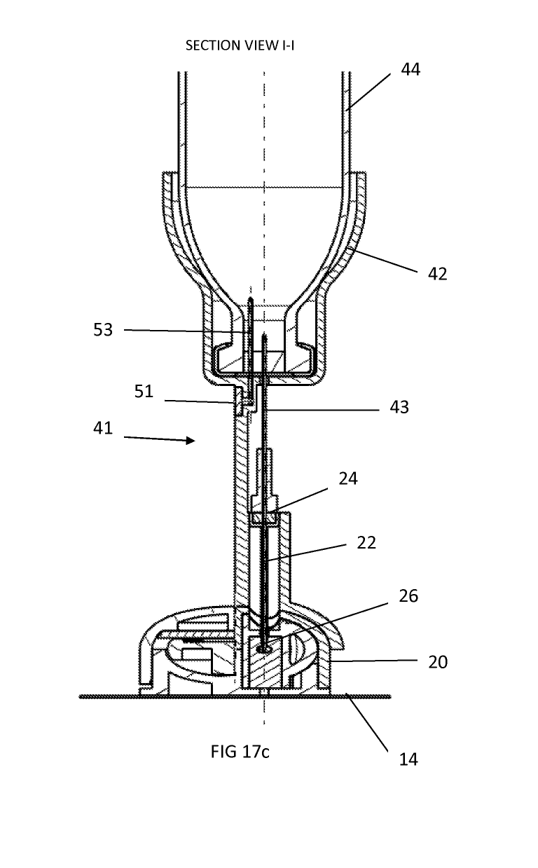

FIG. 17b is section view along line H-H of FIG. 17a, with a view of the pin inside U-shape.

The support 42 for the fluid recipient 44 may also support a second, fixed needle whose upper end communicates with the inside of a supported fluid recipient and whose lower end is open to the ambient air. Such an embodiment is shown in FIG. 17c which is a section view along line I-I of FIG. 17a, showing that the bi-functional connector 41 has optionally a second needle 53 fixed on it. The upper extremity of the fixed needle 53 is preferably positioned higher than the movable needle 43. The upper extremity of the fixed needle 53 is in contact with drug or air in the recipient 44. Its second, lower extremity is in contact with ambient air preferably through a hydrophobic membrane 51. When the piston 38 is moving back, the drug in the recipient 44 is sucked through the movable needle 43, then passes through the septum hole 26 and goes inside the cylinder's inlet/outlet port and fills the cylinder 28. During this filling phase, a vacuum is generated inside the drug recipient 44. This vacuum will exercise a counter pressure resulting in a counterforce on the piston 38 requiring more energy to fill the cylinder 28. To avoid this vacuum during filling, the fixed needle 53 creates an air vent allowing ambient air to go inside the recipient 44 to compensate the inside pressure of the recipient 44. The fixed needle 53 also allows to equilibrate the inside pressure of the drug recipient 44 with ambient air when connecting the recipient 44 to the bi-functional connector 41. This is particularly useful when the patient travels in an airplane, or changes altitude between the last use of the drug recipient. Uncompensated pressure in the drug recipient 44 could result in over or under-pressure during setup and create leakage through septum and incorrect filling.

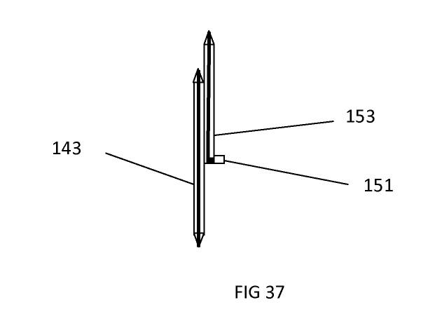

In another embodiment presented on FIG. 37, the movable needle 143 can have a second channel/needle 153. The upper extremity of the second channel is preferably positioned above the upper extremity of the first channel of the needle 143 and the lower extremity of the second channel 153 is in contact with ambient air, optionally through a hydrophobic element 151 arranged to form an air vent.

The needle 53's hydrophobic membrane 51, 151 ensures that no drug will go outside and maintains the bi-functional connector clean. The upper extremity of the fixed needle 53 is preferably higher than the movable needle 43 to avoid ambient air that goes inside the recipient from being sucked with the drug during the filling phase.

### Multi-Function Remote Control

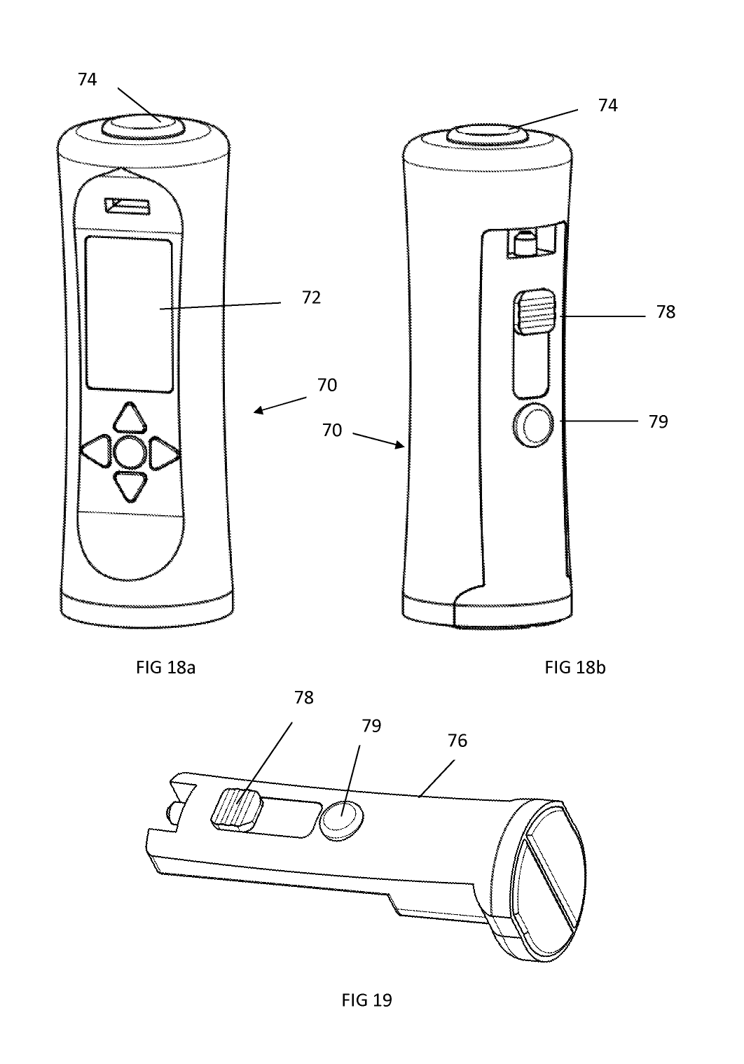

FIGS. 18a and 18b show a first embodiment of an “all-in-one” multi-function remote control 70.

The multi-function remote control 70 can have a glucose sensor that is optionally removable, a display that is optionally a touch screen display 72, optional keypads, optionally at least one activating button 74 preferably placed on the upper side, and optionally at least one compartment.

The multi-function remote control 70 can have a lancing device 76 that is optionally removable. The lancing device 76 preferably has an activating means 78 and a release button 79. FIG. 19 is a view of the lancing device having preferably at least one compartment for disposable lancets and preferably one compartment for disposable glucose strips.

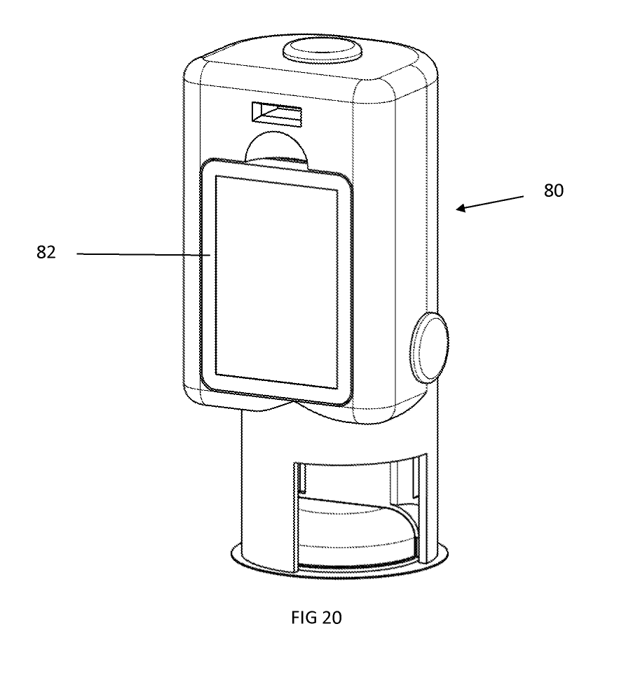

FIG. 20 shows a second embodiment of multi-function remote control 80 having a preferably removable control unit 82 with a preferred touch screen. The control unit has electronic circuitry, a CPU, a memory, power management, preferably at least one rechargeable battery, preferably an interface with a glucose sensor or bio-analytical sensors, preferably multi-axis accelerometers, a wired or wireless interface with the drive unit of the fluid pumping device, a wired or wireless interface with an external device such as a smartphone, PC, tablet, keypad, earphones or any other type of device or sensor, optionally a sound speaker, optionally a vibrator and optionally at least one visual indicator. One control unit 82 is able to control multiple drive units. Each drive unit is paired with the control unit following a preferably secured and encrypted protocol to avoid hacking or interferences of the communication links between the remote unit and the drive unit.

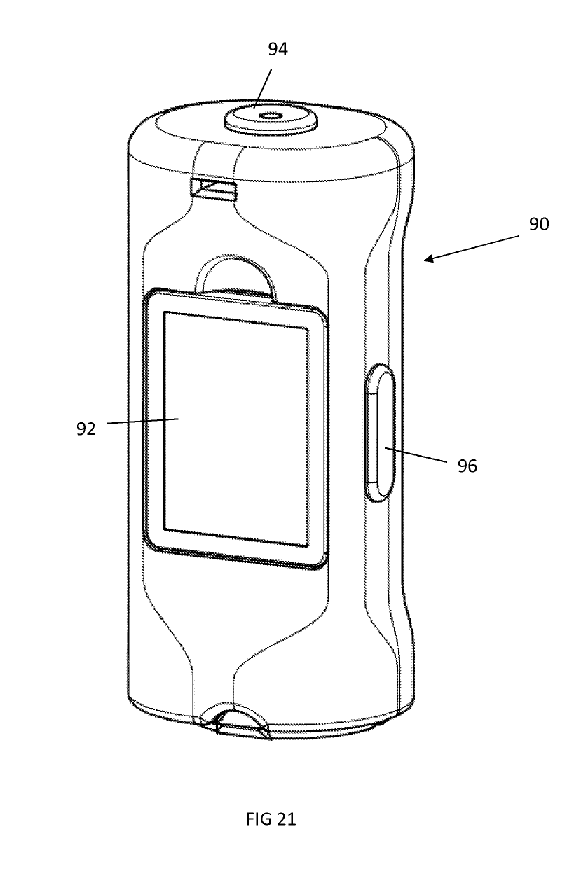

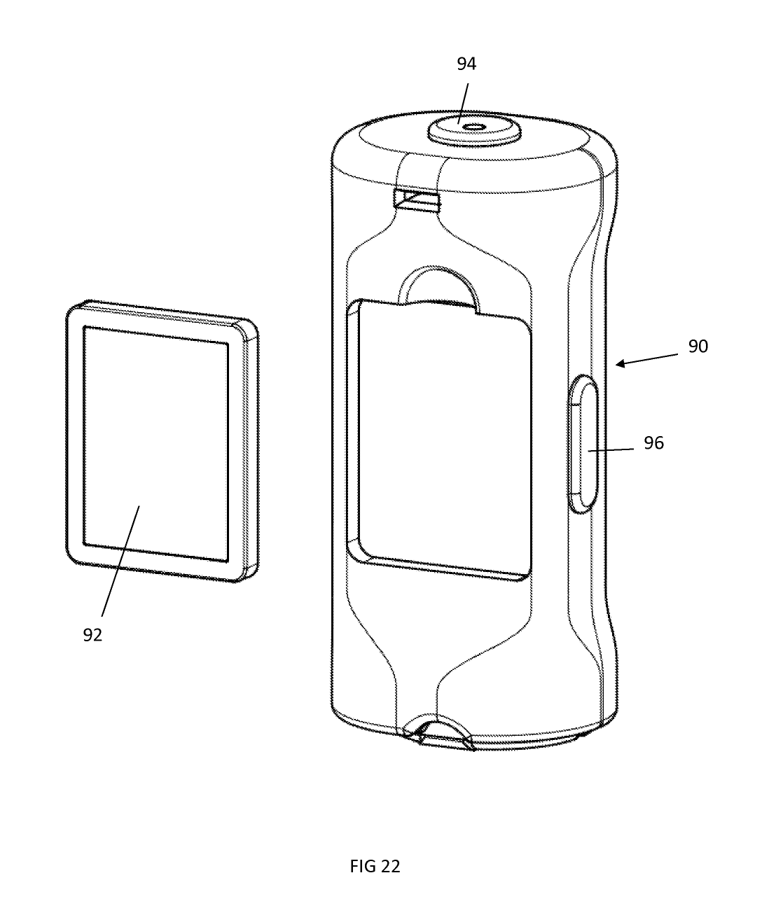

FIGS. 21 and 22 show a third embodiment of multi-function remote control 90 with a removable control unit 92 and top and side control buttons 94, 96.

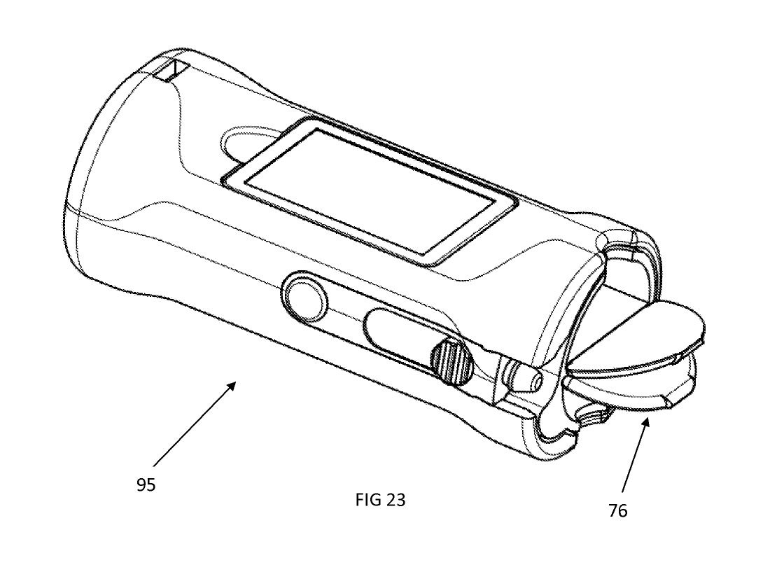

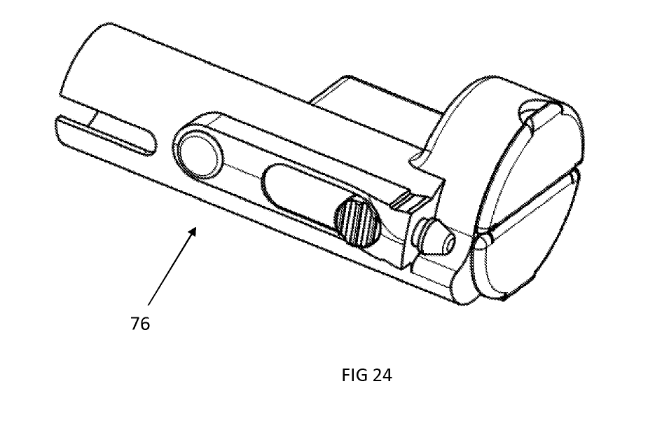

FIG. 23 shows a remote control 95 incorporating a removable lancing device 76 as well as a compartment for lancets and a compartment for test strips. FIG. 24 shows the removable lancing device 76 with an optional compartment.

The multi-functions remote control 90 has optionally at least one button 94 on the upper side, optionally at least one button 96 on one side, optionally a slot for receiving a drive unit, optionally a plug for recharging the drive unit, optionally a drug recipient level sensor, optionally an actuator means for releasing the movable needle support of the bi-functional connector, an optional latch to maintain the disposable housing and/or the bi-functional connector, optionally at least one sensor for detecting the insertion of the disposable housing with the bi-functional connector, the drive unit and the drug recipient, and an optional barcode or RFID reader to automatically detect the drug type.

The control unit has a programmable system for managing drug delivery profiles such as hourly, daily, weekly, monthly and yearly basal rate presets, hourly, daily, weekly monthly and yearly bolus volume, custom profiles, drug volume calculator; glucose control; carbohydrate calculator; a drug library; a library of food parameters such as calories, sugars, hydrates, proteins, vitamins, nutrients, fats; patient physiological parameters such as weight, age, sex, physical conditions, illness, patient activity; time, date. The control unit is also programmable to setup the volume for automatic filling, parameters of the filling procedure such as flow rate, time, viscosity, type of drug, and timing for releasing the movable needle support after button activation, drug recipient level.

The control unit can have an optional integrated or removable sensor for glucose measurement or any other bio-analytic parameter.

The remote control can have an optional slot for receiving a drive unit.

The remote control can be adapted to recharge the drive unit battery by direct electrical contact or by electromagnetic induction.

The remote control or control unit can be adapted to measure the weight of a drug recipient in order to calculate the remaining volume of drug in the recipient and automatically manage the parameters of the filling procedure of the fluid pumping device.

The remote control can also be adapted to manage two or more drive units alternatively for a continuous treatment.

The drive units used for the treatment can be synchronized directly, or by the remote control, or the remote unit or any other external device in order to have the same or appropriate functional settings and drug delivery configurations.

### Method of Use

The filling of the cylinder 28 is preferably done using the remote control 70, 80, 90, after assembling the disposable housing 20 with the bi-functional connector 41 and the drug recipient 44. Once assembled, the patient activates the automatic filling either by preset volume or manual volume. The filling is preferably activated if the disposable housing 20 of the system is horizontally positioned. Multi-axis accelerometers of the drive unit or remote control indicate the position of the system before and during the filling. If the system moves, rotates or falls before or during the filling procedure, the control unit stops the filling.

The filling is preferably completed after a priming to remove air in the drug pathway. Once completed, an alert/signal is either visually or audibly emitted to indicate the status of the filling. The operator/patient is optionally requested to validate the next step and pre-activation of automatic release of the movable needle support.

The operator/patient then takes the multi-functions remote control 70, 80, 90 in hand, removes the adhesive protection on support 14 and applies the support against the patient's skin. Then when ready, the operator/patient presses one of the activating buttons on the multi-functions remote control 70, 80, 90. The electronic circuitry or a mechanical element then engages the activator means that releases the movable needle support 47. The needle 43 pierces the patient's skin, places the cannula 22 under the skin and retracts to its final position, as described above. The latch of the multi-function remote control 70, 80, 90 releases the fluid pumping device that is in place for delivering the drug.

The drive unit 30 or the control unit can be programmed to stop the drug delivery under certain conditions such as period of time, patient activity status, system failure, shocks, communication interferences and others situations.

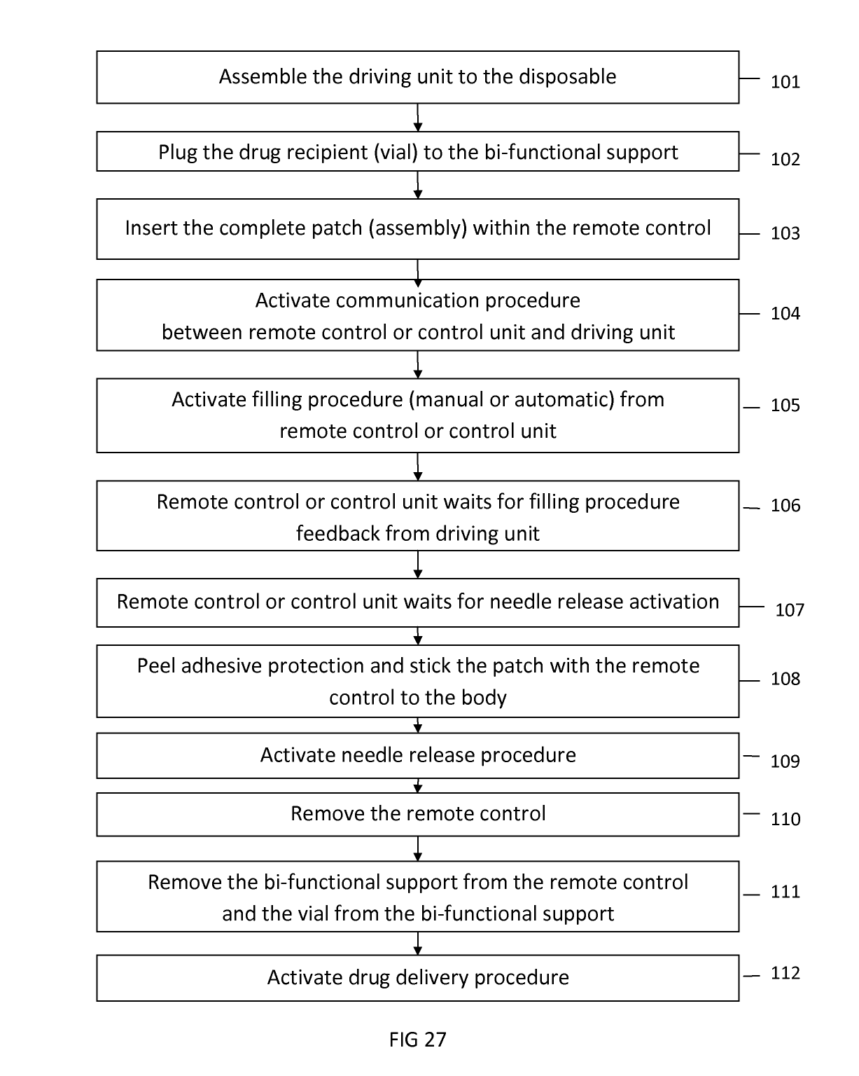

FIG. 27 is a flow diagram of one embodiment of the successive operating steps for installing and using the fluid delivery device according to the invention. According to this protocol, the user begins in step 101 by assembling the drive unit 30 to the disposable unit, i.e. housing 20. In the next step 102 the drug recipient 44 (vial) is plugged into the bi-functional support 41. Then in step 103 the complete patch i.e. the drive unit 30 assembled with the disposable housing 20, is inserted within the remote control unit 70, 80, 90. Next in step 104 the communication procedure between the remote control and the drive unit is activated. In step 105 the manual or automatic filing procedure is activated from the remote control 70, 80, 90 or control unit 82, 92. Next, at step 106, the remote control 70, 80, 90 or control unit 82, 92 waits for feedback on the filling procedure from the drive unit 30, then in step 107 the remote control or control unit waits for activation of the release of needle 43. The user then removes the peel-off protective layer from support 14 and sticks the patch (disposable housing 20 and drive unit 30) with the remote control 70, 80, 90 to the patient's body, in step 108. The needle release procedure is activated in step 109, then the remote control 70, 80, 90 is removed (step 110). Next, the bi-functional support 41 is removed from the remote control unit 70, 80, 90 and the vial (drug recipient 44) is removed from the bi-functional support 41. Lastly, in step 112, the drug delivery procedure is activated so that the drug (eg insulin) is delivered in controlled manner to the patient by driving the piston 38 at a controlled rate.

Steps 101 and 102 can be made in the inverse order.

Steps 103, 110 and 111 are optional when the filling is made without insertion of the assembly in the remote control.

Step 108 can be done by applying the patch to the skin directly with the assembly in hand, without the use of the remote control.

Steps 110 and 111 can be reordered after step 112.

The drug recipient/vial can optionally be removed after the filling, before the needle release activation.

Each procedure is preferably using a double hand check protocol to confirm a proper communication between the control unit and the drive unit.

Each procedure can be programmed and modified manually by the user or automatically updated with an external device such as PC, mobile device or any other electronic device.

The filling procedure can be either manually controlled by the user or preset to automatically fill the drug reservoir. The preset procedure can be optionally programmed to follow a custom filling profile with different parameters such as multiple filling speeds, volume increments, piston directions and pause times. A position sensor inside the driving detects if the patch and remote is correctly positioned (vertical).

The filling procedure and/or profile can be adapted to the drug type, drug reservoir type, drug viscosity and filling conditions such as temperature, vibrations, system position or orientation, drug level in recipient.

The control unit and the drive unit can be adapted to store multiples filling procedures and multiples profiles.

The filling procedure and/or profile can be entirely or partially stored in the control unit memory or in the drive unit memory.

The needle release procedure can be either activated by the control unit sending command to the release mechanism of the multi-functions remote control or adapted to the manual activation of the needle insertion.

### Second Embodiment of Bi-Functional Connector

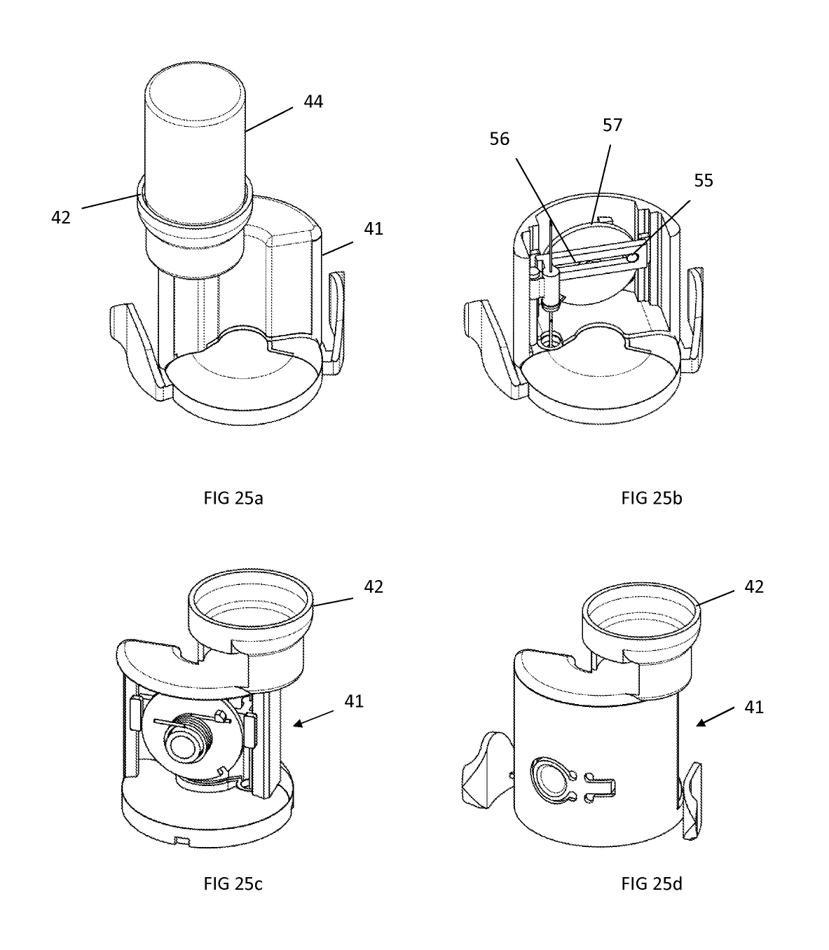

FIGS. 25a, 25b, 25c and 25d show a second embodiment of the bi-functional connector 41 with a rotary element moving a pin in a groove replacing the U-shape. See in particular FIG. 25b which illustrates the rotary element 57 moving a pin 55 in a groove 56. The disposable housing 20 is then, after removing the peel-off layer protecting the adhesive on the support, placed manually onto the patient's skin. The movable needle support 47 is manually activated by pushing on the release mechanism at the back side of the bi-functional support 41. The bi-functional support 41 is then released manually by pressing the preferably lateral latch.

### Variations

The filling of the cylinder 28 can be remotely operated without assembling the multi-functions remote control 70, 80, 90 to the disposable housing 20.

The control unit can be a mobile phone, smartphone or a watch.

The sensors of the fluid delivery system as described in any embodiment can be directly or indirectly in communication with the fluid path.

The electronic circuitry can be adapted to interface with any type of external sensor.

The electronic circuitry can be adapted to transfer energy form the remote control or the control unit to the drive unit during working procedures such as but not limited to the filling phase of the reservoir, data communication and battery recharging.

The communication protocol between the drive unit of the fluid pumping device and the control unit can be of any type. Either the drive unit or the control unit can be programmed in order to adapt the fluid delivery accordingly to the patient inputs or sensor(s) data.

Seal elements of the fluid pumping device according to any embodiment of the invention can be any sort of O-ring and/or any specific gasket. Besides, any part of the fluid pumping device can be machined or obtained by an injecting molding/over molding process. The cylinder can also be made of glass, ceramic or having special coating for not altering the drug during storage.

Although the fluid delivery system as described in the different embodiments of the invention is particularly adapted to be used as an insulin pump, its essential components can also be scaled up to any size so that the fluid delivery system can operate in other fields. For instance, a high-pressure resistance fluid delivery system operating over a wide range of flow rates can be obtained. The fluid delivery system can also be prefilled at the manufacturing site, to avoid the filling process by the user.

In a non-illustrated embodiment, the reservoir/cylinder can be adapted to be filled by means of a syringe for filling the reservoir without a drug recipient or for adding another liquid to the drug. The filling procedure can be adapted to such conditions.

In another non-illustrated embodiment, the bi-functional connector can be reduced to a simple cannula inserter with no drug recipient connector.

In another non-illustrated embodiment, the bi-functional connector can have only part of the components above described and the other components are integrated in the remote control or any other device. As for example, the needle and the recipient support can be part of the bi-functional connector and the activation mechanism is integrated in a separate reusable or disposable device. In another variation, only the needle is part of the bi-functional connector.

In another non-illustrated embodiment, the bi-functional connector can be adapted to be a fully disposable applicator for manual placement of the patch on the patient skin.

The hydrophobic membrane 51, 151 can be replaced by a least one micro hole in the needle.

The hole(s) is/are dimensioned as to allow only the air to pass through while avoiding leakage of the drug.

The disposable housing can be adapted to allow seeing the cylinder/reservoir and piston with an opening or by using semi or fully transparent material. The disposable housing and/or the cylinder/reservoir can be graduated by any means.

The disposable housing can be adapted to integrate a sensor such as, but not limited to, a glucose sensor. The sensor can optionally have a means to pierce the skin to access the tissue on or under the skin such as, but not limited to, a needle or micro needle. The sensor can optionally be placed simultaneously with the cannula 22. The sensor can have electrical connector(s) positioned on the disposable housing to be in contact with electrical connectors(s) on the drive unit for transmitting to the electronic circuitry. The sensor can also be powered by inductive means and transfer data wirelessly to the electronic circuitry. This configuration is well adapted for making a closed loop system.

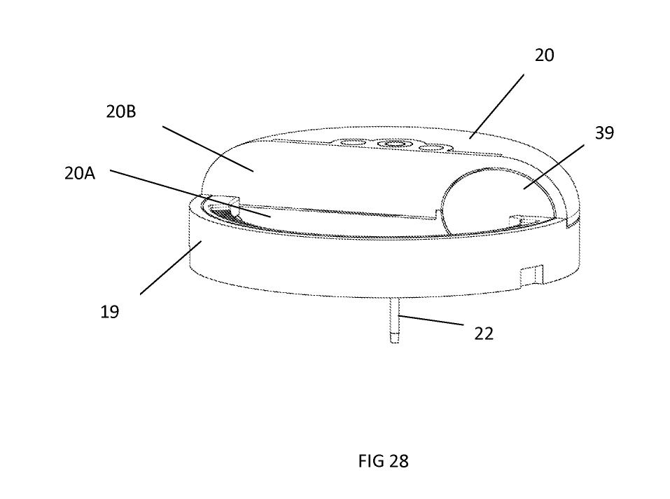

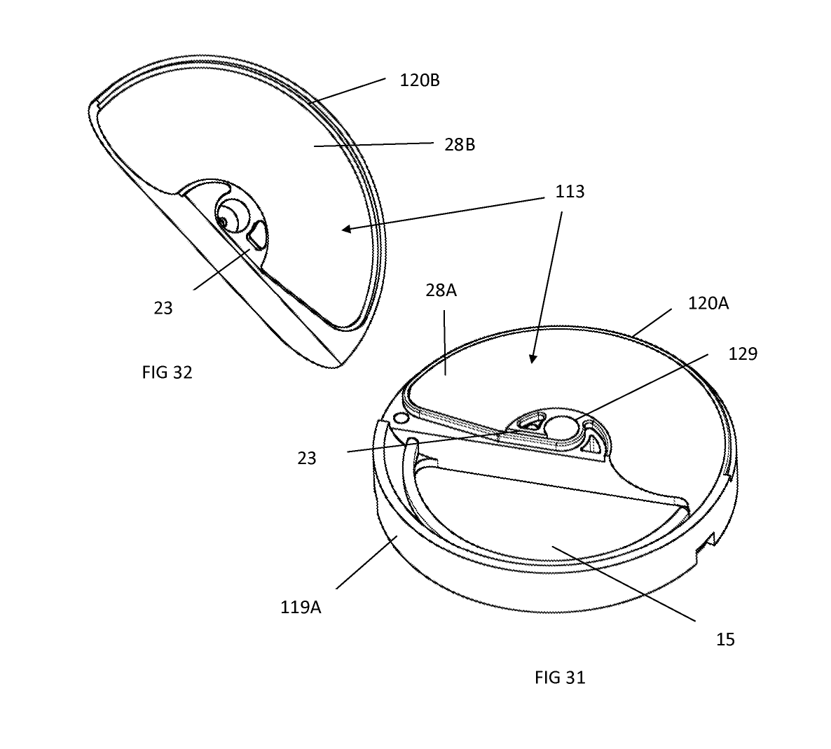

FIGS. 28 to 30 show a disposable patch with its disposable housing 20 and a centered cannula 22. FIGS. 31 and 32 show the two parts/shells 20A/120A and 20B/120B of this patch that make up the disposable housing whereby the inner preferably partial toroidal arcuate cavity 113 forms the cylinder by the assembly of the two half-cylinders 28A and 28B. FIGS. 31, 32 also show a channel 23 by which the cannula 22 (via the septum in its upper head 27) communicates with the arcuate cylinder 28A, 28B. The lower and upper parts/shells 120A, 120B have also preferably an arcuate wall 119A, 119B on one half of its circumference. The septum is placed in the septum support 129.

The upper part/shell 20B or 120B has preferably a projected surface in the form of half a disc.

The two parts/shell 120A, 120B can be adapted to support the rotatable element 251 of the piston 138 in a similar configuration as the fixed support 252A and 252B positioned on the two parts 20A,20B.

The two parts/shell 20A,20B or 120A/120B of the disposable housing are preferably joined at the central section plan B-B of the housing or cylinder, making each part one half of the arcuate cavity 13, 113 or cylinder 28.

The two parts/shells 20A, 20B or 120A, 120B of the disposable housing are preferably attached together by ultrasonic welding, gluing or clipping/snap fit means.

The cannula 22 is preferably passing through the two parts/shells 20A, 20B or 120A, 120B of the disposable housing.

Elements and/or features of different illustrative embodiments may be combined with each other and/or substituted for each other within the scope of this disclosure and appended claims.

For convenience the references numbers and their corresponding features are listed in the following legend:

LEGEND

| | |
|---|---|
| 10. | Fluid delivery device |
| 12. | Diametral dividing line |
| 13, 113. | Arcuate cavity |
| 14. | Adhesive support |
| 15. | Recess |
| 19A, 19B. | Wall |
| 20, 120. | Disposable housing unit |
| 20A, 20B. | Parts/Shells |
| 21A, 21B. | Curved profile |
| 22. | Cannula |
| 23. | Channel |
| 24. | Septum |
| 25. | Hole in cannula |
| 26. | Through hole |
| 27. | Upper head |
| 28. | Arcuate cylinder |
| 28A, 28B. | Cylinder parts |
| 29, 129. | Septum support |
| 30, 130. | Drive unit |
| 31. | Motor |
| 32. | Electrical connector |
| 33. | Driving unit case |
| 34. | Motor gear wheel |
| 35. | Motor axis |
| 36, 136. | Serrations, rack |
| 37. | Opening |
| 38, 138. | Piston |
| 39. | Piston head |
| 40, 240. | Stem |
| 41. | Bi-functional connector |
| 42. | Support for 44 |
| 43, 153. | Needle |
| 44. | Drug recipient |
| 45. | Needle grab/clamp |
| 47. | Movable support |
| 48. | Groove |
| 49. | Pin |
| 50. | U-shaped groove |
| 51, 151. | Hydrophobic membrane |
| 52. | Spring |
| 53, 153. | Second needle |
| 54. | Aperture |
| 55. | Pin |
| 56. | Groove |
| 57. | Rotary element |
| 61. | CPU |
| 62. | Power management |
| 63. | Sensor unit |
| 64. | External device interface |
| 65. | Memory |
| 66. | User interface |
| 67. | Audio interface |
| 68. | Actuator driver |
| 70. | Multi-function remote control |
| 72. | Touch screen display |
| 74. | Activating button |
| 76. | Lancing device |
| 78. | Activating means |
| 79. | Release button |
| 80. | 2^{nd} embodiment of remote control |
| 90. | 3^{rd} embodiment of remote control |
| 92. | Removable control unit |
| 94, 96. | Buttons |
| 101-112. | Steps |
| 119A, 119B. | Wall |
| 120A, 120B. | Parts/Shell |
| 250. | Piston support |
| 251. | Rotatable element |
| 252A, 252B. | Axial joint, hinge |
| 255. | Gasket |
| 258, 259. | Sensor element |
| 260. | Stem reinforcement |

1. A fluid delivery device for delivering small quantities of a fluid to a patient, comprising a disposable housing, characterized in that the disposable housing comprises one lower part and one upper part, the upper and lower parts being separate parts that when fitted together form a shell that defines an internal partial toroidal arcuate cavity.

2. The fluid delivery device of claim 1, wherein the arcuate cavity receives an arcuate cylinder for containing the fluid or wherein the arcuate cavity forms an arcuate cylinder for containing the fluid.

3. (canceled)

4. The fluid delivery device of claim 2 or 3, wherein an arcuate piston is movable inside the arcuate cylinder.

5. The fluid delivery device of claim 4, wherein the piston has a support at its bottom which cooperates with at least one support on the upper and/or lower part of the housing.

6. The fluid delivery device of claim 4, wherein the piston has a reinforced stem at its bottom.

7. The fluid delivery device of claim 1, wherein at least one of said lower or upper parts forming the shell of the disposable housing has an arcuate wall on one half of its circumference opposite to the arcuate cavity.

8. The fluid delivery device of claim 4, wherein a removable drive unit comprising means for actuating the piston is attachable to the disposable housing.

9. The fluid delivery device of claim 7,
wherein a removable drive unit comprising means for actuating the piston is attachable to the disposable housing, and said arcuate wall constitutes a support for receiving, fixing and sealing the drive unit to the disposable housing.

10. The fluid delivery device of claim 8, wherein the disposable housing has a recess with outer borders defined by the inner side of the piston stem and by a diametral line of the disposable housing for receiving therein the drive unit.

11. The fluid delivery device of claim 8, wherein the disposable housing is an overall enveloping housing in the form of a generally flat cylindrical disc with rounded or inclined upper edges and a flat bottom, the drive unit occupying about one half of the top surface of the flat cylindrical disc, and wherein an adhesive support is applied against the flat bottom and projects from the flat bottom as a peripheral rim.

12. The fluid delivery device of claim 1, wherein a straight cannula is generally perpendicular to an adhesive support placed under one part/shell of the disposable housing and is located towards the downstream end of the said toroidal cavity.

13. The fluid delivery device of claim 1, wherein a cannula is movably mounted in the disposable housing between a first position for delivering fluid to a patient and a second position communicating the cavity with the outside for filling the cavity or a cylinder therein with fluid from an external recipient.

14. The fluid delivery device of claim 1, wherein a cannula passes through the two parts/shells of the disposable housing.

15. The fluid delivery device of claim 12, wherein the cannula cooperates with a septum having therein an aperture.

16. The fluid delivery device of claim 8, wherein the drive unit is actuable by remote control.

17. The fluid delivery device of claim 1, wherein the lower and upper parts/shells of the disposable housing are fixed by ultrasonic welding, by glue or by clipping/snap fit.

18. (canceled)

19. (canceled)

20. The fluid delivery device of claim 1, wherein the arcuate cavity or a cylinder located in the arcuate cavity contains insulin for delivery to a patient.

21. A system for delivering small quantities of a fluid to a patient, comprising:
a fluid delivery device according to claim 1; and
a bi-functional connector removably fittable on the fluid delivery device, the bi-functional connector comprising a support for a fluid recipient, a movable needle held in a needle grip, the needle being movable when the bi-functional connector is fitted on the fluid delivery device between a position for delivering fluid from the fluid recipient to the cylinder, a position for causing the needle to pierce the patient's skin for inserting the cannula and a position allowing the cylinder to deliver fluid via the cannula.

22. The system of claim 21 which further comprises a remote control that is adapted to communicate wirelessly with the fluid delivery device, the remote control comprising a plurality of controls for different functions of the fluid delivery device.

23. A bi-functional connector removably fittable on the fluid delivery device of claim 1, the bi-functional connector comprising a support for a fluid recipient, a movable needle held in a needle grip, the needle being movable when the bi-functional connector is fitted on the fluid delivery device between a position for delivering fluid from the fluid recipient to the cylinder, a position for causing the needle to pierce the patient's skin for inserting the cannula and a position allowing the cylinder to deliver fluid via the cannula.

24. The bi-functional connector of claim 23, wherein the support for the fluid recipient supports a second, fixed needle whose upper end communicates with the inside of a supported fluid recipient and whose lower end is open to the ambient air.

25-30. (canceled)

31. A method for installing the fluid delivery device of claim 1 on a patient, the method comprising:
adhering the disposable housing to a patient's skin by means of an adhesive support;
fitting a bi-functional support on the fluid delivery device, the bi-functional support carrying a recipient of fluid to be delivered and being adapted to deliver fluid to the fluid delivery device via a needle;
actuating a piston in the arcuate cavity or in a cylinder contained in the arcuate cavity to deliver fluid to the disposable housing's cavity or cylinder; and
actuating the needle to pierce the patient's skin for inserting a cannula and bring the cannula into communication with fluid in the cavity or cylinder for delivering fluid to the patient's body.

32. The method of claim 31, wherein functions of the fluid delivery device are controlled by a remote control that communicates with the fluid delivery device by wireless communication.
